SEQUENCE LISTING (23 sequences)

SEQ ID NO: 1 - protein, 222 aa, Gadus morhua
  Trypsin type I from Atlantic cod
IVGGYECTKHSQAHQVSLNSGYHFCGGSLVSKDWVVSAAHCYKSRIEVRLGEHHIRVNEGTEQYISSSSVIRHPNYSSYNINNDIMLIKLSKPATLNQYVQPVALPTECAADGTMCTVSGWGNTMSSVADGDKLQCLSLPILSHADCANSYPGMITQSMFCAGYLEGGKDSCQGDSGGPVVCNGVLQGVVSWGYGCAERDHPGVYAKVCVLSGWVRDTMANY

SEQ ID NO: 2 - protein, 222 aa, Gadus morhua
  Trypsin type I from Atlantic cod
IVGGYECTKHSQAHQVSLNSGYHFCGGSLVSKDWVVSAAHCYKSVLRVRLGEHHIRVNEGTEQYISSSSVIRHPNYSSYNINNDIMLIKLTKPATLNQYVHAVALPTECAADATMCTVSGWGNTMSSVADGDKLQCLSLPILSHADCANSYPGMITQSMFCAGYLEGGKDSCQGDSGGPVVCNGVLQGVVSWGYGCAERDHPGVYAKVCVLSGWVRDTMANY

SEQ ID NO: 3 - protein, 227 aa, Gadus morhua
  Consensus sequence of ZT isoforms 1-4
  VARIANT 2: Wherein Xaa is Ile or Val
  VARIANT 5: Wherein Xaa is Gln or His
  VARIANT 6: Wherein Xaa is Asp or Glu
  VARIANT 10: Wherein Xaa is Arg or Asn
  VARIANT 77: Wherein Xaa is Leu
  VARIANT 148: Wherein Xaa is Thr or Pro
  VARIANT 150: Wherein Xaa is Asp or Ala
  VARIANT 152: Wherein Xaa is Glu or Gln
  VARIANT 154: Wherein Xaa is Ala or Ser
  VARIANT 164: Wherein Xaa is Val or Met
  VARIANT 166,175: Wherein Xaa is Ala or Val
  VARIANT 168,215: Wherein Xaa is Tyr or Phe
  VARIANT 198: Wherein Xaa is Gln or Arg
  VARIANT 202: Wherein Xaa is Leu or Glu
  VARIANT 205: Wherein Xaa is Tyr or Ser
IXGGXXCEPXSRPFMASLNYGYHFCGGVLINDQWVLSVAHCWYNPYYMQVMLGEHDLRVFEGTEQLVKTNTIFWHEXYDYQTLDYDMMMIKLYHPVEVTQSVAPISLPTGPPDGGMLCSVSGWGNMAMGEEVNLPTRLQCLDVPIVEXVXCXAXYPGMISPRMXCXGXMDGGRDXCNGDSGSPLVCEGVLTGLVSWGXGCAXPNXPGVYVKVYEXLSWIQTTLDANP

SEQ ID NO: 4 - protein, 227 aa, Gadus morhua
  Atlantic cod trypsin ZT-1 isoform
IVGGHECEPNSRPFMASLNYGYHFCGGVLINDQWVLSVAHCWYNPYYMQVMLGEHDLRVFEGTEQLVKTNTIFWHELYDYQTLDYDMMMIKLYHPVEVTQSVAPISLPTGPPDGGMLCSVSGWGNMAMGEEVNLPTRLQCLDVPIVEPVACQASYPGMISPRMMCVGFMDGGRDVCNGDSGSPLVCEGVLTGLVSWGRGCAEPNSPGVYVKVYEFLSWIQTTLDANP

SEQ ID NO: 5 - protein, 227 aa, Gadus morhua
  Atlantic cod trypsin ZT-2 isoform
IVGGHECEPNSRPFMASLNYGYHFCGGVLINDQWVLSVAHCWYNPYYMQVMLGEHDLRVFEGTEQLVKTNTIFWHELYDYQTLDYDMMMIKLYHPVEVTQSVAPISLPTGPPDGGMLCSVSGWGNMAMGEEVNLPTRLQCLDVPIVETVDCEAAYPGMISPRMVCAGYMDGGRDACNGDSGSPLVCEGVLTGLVSWGQGCALPNYPGVYVKVYEYLSWIQTTLDANP

SEQ ID NO: 6 - protein, 227 aa, Gadus morhua
  Atlantic cod trypsin ZT-3 isoform
IIGGQDCEPRSRPFMASLNYGYHFCGGVLINDQWVLSVAHCWYNPYYMQVMLGEHDLRVFEGTEQLVKTNTIFWHELYDYQTLDYDMMMIKLYHPVEVTQSVAPISLPTGPPDGGMLCSVSGWGNMAMGEEVNLPTRLQCLDVPIVEPVACQASYPGMISPRMMCVGFMDGGRDVCNGDSGSPLVCEGVLTGLVSWGRGCAEPNSPGVYVKVYEFLSWIQTTLDANP

SEQ ID NO: 7 - protein, 227 aa, Gadus morhua
  Atlantic cod trypsin ZT-4 isoform
IIGGQDCEPRSRPFMASLNYGYHFCGGVLINDQWVLSVAHCWYNPYYMQVMLGEHDLRVFEGTEQLVKTNTIFWHELYDYQTLDYDMMMIKLYHPVEVTQSVAPISLPTGPPDGGMLCSVSGWGNMAMGEEVNLPTRLQCLDVPIVETVDCEAAYPGMISPRMVCAGYMDGGRDACNGDSGSPLVCEGVLTGLVSWGQGCALPNYPGVYVKVYEYLSWIQTTLDANP

SEQ ID NO: 8 - protein, 222 aa, Gadus morhua
  Atlantic cod trypsin X
IVGGYECTRHSQAHQVSLNSGYHFCGGSLVSKDWVVSAAHCYKSVLRVRLGEHHIRVNEGTEQFISSSSVIRHPNYSSYNIDNDIMLIKLTEPATLNQYVHAVALPTECAADATMCTVSGWGNTMSSVDDGDKLQCLNLPILSHADCANSYPGMITQSMFCAGYLEGGKDSCQGDSGGPVVCNGVLQGVVSWGYGCAERDNPGVYAKVCVLSGWVRDTMASY

SEQ ID NO: 9 - protein, 222 aa, Gadus morhua
  Atlantic cod trypsin X-1
IVGGYECTRHSQAHQVSLNSGYHFCGGSLVSKDWVVSAAHCYKSRIEVRLGEHHIRVNEGTEQFISSSSVIRHPNYSSYNIDNDIMLIKLSEPATLNQYVQPVALPTECAADGTMCTVSGWGNTMSSVDDGDKLQCLNLPILSHADCANSYPGMITQSMFCAGYLEGGKDSCQGDSGGPVVCNGVLQGVVSWGYGCAERDNPGVYAKVCVLSGWVRDTMASY

SEQ ID NO: 10 - protein, 222 aa, Gadus morhua
  Atlantic cod trypsin X-2
IVGGYECTRHSQAHQVSLNSGYHFCGGSLVSKDWVVSAAHCYKSRIEVRLGEHHIRVNEGTEQFISSSSVIRHPNYSSYNIDNDIMLIKLSKPATLNQYVQTVALPTECAADGTMCTVSGWGNTMSSVDDGDKLQCLNLPILSHADCSNSYPGMITQSMFCAGYLEGGKDSCQGDSGGPVVCNGVLQGVVSWGYGCAERDNPGVYAKVCVLSGWVRDTMASY

SEQ ID NO: 11 - protein, 222 aa, Gadus morhua
  Atlantic cod trypsin X-3
IVGGYECTRHSQAHQVSLNSGYHFCGGSLVSKDWVVSAAHCYKSRIEVRLGEHHIRVNEGTEQFISSSSVIRHPNYSSYNIDNDIMLIKLSEPATLNQYVQTVALPTECAADGTMCTVSGWGNTMSSVDDGDKLQCLNLPILSHADCSNSYPGMITQSMFCAGYLEGGKDSCQGDSGGPVVCNGVLQGVVSWGYGCAERDNPGVYAKVCVLSGWVRDTMASY

SEQ ID NO: 12 - protein, 227 aa, Gadus morhua
  Atlantic cod trypsin Y
IIGGQDCEPRSRPFMASLNYGYHFCGGVLINDQWVLSVAHCWYNPYYMQVMLGEHDLRVFEGTEQLVKTNTIFWHEQYDYQTLDYDMMMIKLYHPVEVTQSVAPISLPTGPPDGGMLCSVSGWGNMAMGEEVNLPTRLQCLDVPIVETVDCEAAYPGMISPRMVCAGYMDGGRDACNGDSGSPLVCEGVLTGLVSWGQGCALPNYPGVYVKVYEYLSWIQTTLDANP

SEQ ID NO: 13 - protein, 241 aa, Gadus morhua
  GenBank accession no. ACO90397.1
MKSLIFVLLLGAVFAEEDKIVGGYECTKHSQAHQVSLNSGYHFCGGSLVSKDWVVSAAHCYKSRIEVRLGEHHIRVNEGTEQYISSSSVIRHPNYSSYNINNDIMLIKLSKPATLNQYVQPVALPTECAADGTMCTVSGWGNTMSSVADGDKLQCLSLPILSHADCANSYPGMITQSMFCAGYLEGGKDSCQGDSGGPVVCNGVLQGVVSWGYGCAERDHPGVYAKVCVLSGWVRDTMANY

SEQ ID NO: 14 - protein, 241 aa, Gadus morhua
  UniProt accession no. P16049-1
MKSLIFVLLLGAVFAEEDKIVGGYECTKHSQAHQVSLNSGYHFCGGSLVSKDWVVSAAHCYKSVLRVRLGEHHIRVNEGTEQYISSSSVIRHPNYSSYNINNDIMLIKLTKPATLNQYVHAVALPTECAADATMCTVSGWGNTMSSVADGDKLQCLSLPILSHADCANSYPGMITQSMFCAGYLEGGKDSCQGDSGGPVVCNGVLQGVVSWGYGCAERDHPGVYAKVCVLSGWVRDTMANY

SEQ ID NO: 15 - protein, 241 aa, Gadus morhua
  GenBank accession no. Q91041.2
MKSLIFVLLLGAVFAEEDKIVGGYECTRHSQAHQVSLNSGYHFCGGSLVSKDWVVSAAHCYKSVLRVRLGEHHIRVNEGTEQFISSSSVIRHPNYSSYNIDNDIMLIKLTEPATLNQYVHAVALPTECAADATMCTVSGWGNTMSSVDDGDKLQCLNLPILSHADCANSYPGMITQSMFCAGYLEGGKDSCQGDSGGPVVCNGVLQGVVSWGYGCAERDNPGVYAKVCVLSGWVRDTMASY

SEQ ID NO: 16 - protein, 241 aa, Gadus morhua
  GenBank accession no. AOX15769.1
MKSLIFVLLLGAVFAEEDKIVGGYECTRHSQAHQVSLNSGYHFCGGSLVSKDWVVSAAHCYKSRIEVRLGEHHIRVNEGTEQFISSSSVIRHPNYSSYNIDNDIMLIKLSEPATLNQYVQPVALPTECAADGTMCTVSGWGNTMSSVDDGDKLQCLNLPILSHADCANSYPGMITQSMFCAGYLEGGKDSCQGDSGGPVVCNGVLQGVVSWGYGCAERDNPGVYAKVCVLSGWVRDTMASY

SEQ ID NO: 17 - protein, 241 aa, Gadus morhua
  GenBank accession no. AOX15770.1
MKSLIFVLLLGAVFAEEDKIVGGYECTRHSQAHQVSLNSGYHFCGGSLVSKDWVVSAAHCYKSRIEVRLGEHHIRVNEGTEQFISSSSVIRHPNYSSYNIDNDIMLIKLSKPATLNQYVQTVALPTECAADGTMCTVSGWGNTMSSVDDGDKLQCLNLPILSHADCSNSYPGMITQSMFCAGYLEGGKDSCQGDSGGPVVCNGVLQGVVSWGYGCAERDNPGVYAKVCVLSGWVRDTMASY

SEQ ID NO: 18 - protein, 241 aa, Gadus morhua
  GenBank accession no. AOX15771.1
MKSLIFVLLLGAVFAEEDKIVGGYECTRHSQAHQVSLNSGYHFCGGSLVSKDWVVSAAHCYKSRIEVRLGEHHIRVNEGTEQFISSSSVIRHPNYSSYNIDNDIMLIKLSEPATLNQYVQTVALPTECAADGTMCTVSGWGNTMSSVDDGDKLQCLNLPILSHADCSNSYPGMITQSMFCAGYLEGGKDSCQGDSGGPVVCNGVLQGVVSWGYGCAERDNPGVYAKVCVLSGWVRDTMASY

SEQ ID NO: 19 - protein, 249 aa, Gadus morhua
  GenBank accession no. CAD30563.1
MIGLALLMLLGAAAAVPREDGRIIGGQDCEPRSRPFMASLNYGYHFCGGVLINDQWVLSVAHCWYNPYYMQVMLGEHDLRVFEGTEQLVKTNTIFWHEQYDYQTLDYDMMMIKLYHPVEVTQSVAPISLPTGPPDGGMLCSVSGWGNMAMGEEVNLPTRLQCLDVPIVETVDCEAAYPGMISPRMVCAGYMDGGRDACNGDSGSPLVCEGVLTGLVSWGQGCALPNYPGVYVKVYEYLSWIQTTLDANP

SEQ ID NO: 20 - protein, 250 aa, Gadus morhua
  Uncleaved Atlantic cod ZT-1
MIGLALLMLLGAAAAAVPRDVGKIVGGHECEPNSRPFMASLNYGYHFCGGVLINDQWVLSVAHCWYNPYYMQVMLGEHDLRVFEGTEQLVKTNTIFWHELYDYQTLDYDMMMIKLYHPVEVTQSVAPISLPTGPPDGGMLCSVSGWGNMAMGEEVNLPTRLQCLDVPIVEPVACQASYPGMISPRMMCVGFMDGGRDVCNGDSGSPLVCEGVLTGLVSWGRGCAEPNSPGVYVKVYEFLSWIQTTLDANP

SEQ ID NO: 21 - protein, 250 aa, Gadus morhua
  Uncleaved Atlantic cod ZT-2
MIGLALLMLLGAAAAAVPRDVGKIVGGHECEPNSRPFMASLNYGYHFCGGVLINDQWVLSVAHCWYNPYYMQVMLGEHDLRVFEGTEQLVKTNTIFWHELYDYQTLDYDMMMIKLYHPVEVTQSVAPISLPTGPPDGGMLCSVSGWGNMAMGEEVNLPTRLQCLDVPIVETVDCEAAYPGMISPRMVCAGYMDGGRDACNGDSGSPLVCEGVLTGLVSWGQGCALPNYPGVYVKVYEYLSWIQTTLDANP

SEQ ID NO: 22 - protein, 249 aa, Gadus morhua
  Uncleaved Atlantic cod ZT-3
MIGLALLMLLGAAAAVPREDGRIIGGQDCEPRSRPFMASLNYGYHFCGGVLINDQWVLSVAHCWYNPYYMQVMLGEHDLRVFEGTEQLVKTNTIFWHELYDYQTLDYDMMMIKLYHPVEVTQSVAPISLPTGPPDGGMLCSVSGWGNMAMGEEVNLPTRLQCLDVPIVEPVACQASYPGMISPRMMCVGFMDGGRDVCNGDSGSPLVCEGVLTGLVSWGRGCAEPNSPGVYVKVYEFLSWIQTTLDANP

SEQ ID NO: 23 - protein, 249 aa, Gadus morhua
  Uncleaved Atlantic cod ZT-4
MIGLALLMLLGAAAAVPREDGRIIGGQDCEPRSRPFMASLNYGYHFCGGVLINDQWVLSVAHCWYNPYYMQVMLGEHDLRVFEGTEQLVKTNTIFWHELYDYQTLDYDMMMIKLYHPVEVTQSVAPISLPTGPPDGGMLCSVSGWGNMAMGEEVNLPTRLQCLDVPIVETVDCEAAYPGMISPRMVCAGYMDGGRDACNGDSGSPLVCEGVLTGLVSWGQGCALPNYPGVYVKVYEYLSWIQTTLDANP

(19) United States
(12) Patent Application Publication
(10) Pub. No.: US 2020/0353060 A1
(43) Pub. Date: Nov. 12, 2020

(54) PEPTIDES HAVING PROTEASE ACTIVITY FOR USE IN THE TREATMENT OR PREVENTION OF CORONAVIRUS INFECTION

(71) Applicant: ENZYMATICA AB, Lund (SE)

(72) Inventors: Ágústa GUDMUNDSDÓTTIR, Reykjavik (IS); Bjarki STEFÁNSSON, Reykjavik (IS); Gunnar Birgir SANDHOLT, Reykjavik (IS)

(21) Appl. No.: 16/960,387

(22) PCT Filed: Jan. 7, 2019
(86) PCT No.: PCT/EP2019/050266

§ 371 Date: Jul. 7, 2020

(30) Foreign Application Priority Data
Jan. 8, 2018 (GB) 1800274.1

(51) Int. Cl. A61K 38/48; A61P 11/00; A61P 31/14

(52) U.S. Cl. CPC A61K 38/4826; A61K 45/06; A61P 31/14; A61P 11/00

(57) ABSTRACT

The present invention provides a polypeptide having protease activity for use in the treatment or prevention of coronavirus infection in a mammal. In particular, the invention relates to treatment or prevention of a coronavirus infection in a human, using trypsins.

### FIELD OF INVENTION

The present invention relates to the use of polypeptides with protease activity for the treatment and prevention of a coronavirus infection in a subject. In particular, the invention relates to treatment or prevention of a coronavirus infection in a mammal using a trypsin (such as marine-derived trypsins, e.g. from Atlantic cod).

### BACKGROUND

Atlantic cod (Gadus morhua) trypsin has been shown in vitro to decrease the infectivity of viruses such as human rhinovirus, respiratory syncytial virus and influenza virus (Fornbacke and Clarsund 2013, Gudmundsdottir, Hilmarsson et al. 2013). This is thought to be partly based on its high catalytic efficiency compared to analogous enzymes (Asgeirsson, Fox et at 1989, Asgeirsson and Cekan 2006, Stefansson, Helgadottir et al. 2010). Trypsins are classified into three main groups, I, II and III based on their deduced amino acid sequence identities (Spilliaert and Gudmundsdottir 1999). The recently identified cod trypsin ZT (see WO 2017/017012 and unpublished data in a manuscript submitted for publication: Sandholt G B, Stefansson B, Gudmundsdottir Á) is a member of group III trypsins whereas cod trypsins I and X belong to group I, like many other trypsins (Asgeirsson, Fox et al. 1989, Gudmundsdottir, Gudmundsdottir et al. 1993, Stefansson, Helgadottir et al. 2010, Stefansson, Sandholt et al. 2017).

Coronaviruses are species of virus belonging to the subfamily Coronavirinae in the family Coronaviridae, in the order Nidovirales. Coronaviruses are enveloped viruses with a positive-sense single-stranded RNA genome and with a nucleocapsid of helical symmetry. The genomic size of coronaviruses ranges from approximately 26 to 32 kilobases, the largest for an RNA virus. The name “coronavirus” is derived from the Latin corona, meaning crown or halo, and refers to the characteristic appearance of virions under electron microscopy (E.M.) with a fringe of large, bulbous surface projections creating an image reminiscent of a royal crown or of the solar corona. This morphology is created by the viral spike (S) peplomers, which are proteins that populate the surface of the virus and determine host tropism. Proteins that contribute to the overall structure of all coronaviruses are the spike (S), envelope (E), membrane (M) and nucleocapsid (N) proteins. In the specific case of the SARS coronavirus (see below), a defined receptor-binding domain on S mediates the attachment of the virus to its cellular receptor, angiotensin-converting enzyme 2 (ACE2). Some coronaviruses (specifically the members of Betacoronavirus subgroup A) also have a shorter spike-like protein called hemagglutinin esterase (HE).

Coronaviruses primarily infect the respiratory tract and gastrointestinal tract of mammals and birds. They are known to be one of the infective agents underlying the common cold, along with rhinoviruses, influenza and respiratory syncytial viruses (Heikkinen and Jarvinen 2003, Hull, Rennie et al. 2007). Approximately 10-15% of common cold cases are estimated to be caused by coronaviruses (Hull, Rennie et al. 2007, Jonsdottir and Dijkman 2016). Four of the six known coronaviruses in rotation cause general upper respiratory illnesses and cold in healthy individuals (i.e. 229E, OC43, NL63 and HKU1) (Bradburne, Bynoe et al. 1967, Kraaijeveld, Reed et al. 1980). The other two coronaviruses are the Severe Acute Respiratory Syndrome (SARS) coronavirus and Middle East Respiratory Syndrome (MERS) coronavirus which can cause severe pneumonia (Park, U et al. 2016). Coronavirus infection begins with attachment of the spike protein with its cognate cell receptor (Jonsdottir and Dijkman 2016, Lim, Ng et al. 2016). SARS-CoV and MERS-CoV, are known to infect epithelial cells by using their spike proteins for cell entry (Park, Li et al. 2016). Cell tropism and host range of coronaviruses is in part determined by the coronavirus spike (S) protein, which binds cellular receptors and mediates membrane fusion (Graham and Baric 2010).

MERS and SARS are on the top of WHO's list of disease priorities needing urgent research due to the high likelihood of causing a major epidemic (WHO). The epidemic potential of the CoVs family emphasizes the need for vaccines and antivirals. Today, no general therapy exists to treat coronavirus induced disease in humans and no commercial vaccines are available (Jonsdottir and Dijkman 2016). Therefore, it is important to find new therapies capable of deactivating coronaviruses.

Thus, the present invention seeks to address the need for new therapies for treating and preventing infection with coronavirus.

### SUMMARY OF INVENTION

The first aspect of the invention provides a polypeptide having protease activity for use in the treatment or prevention of coronavirus infection in a subject, such as a mammal or bird.

By “polypeptide having protease activity” we include any polypeptide (both naturally occurring and non-naturally occurring) which is capable of catalysing proteolysis in vivo, in the mammalian (e.g. human) body. Thus, any type of protease may be utilised in the invention, including but not limited to serine proteases (such as trypsins/chymotrypsins), threonine proteases, cysteine proteases, aspartate proteases, glutamic acid proteases and metalloproteases.

By “coronavirus infection” we mean an infection caused by or otherwise associated with growth of coronavirus in a subject, in the family Coronaviridae (subfamily Coronavirinae).

By “treatment” we include the alleviation, in part or in whole, of the symptoms of coronavirus infection (e.g. the sore throat, blocked and/or runny nose, cough and/or elevated temperature associated with a common cold). Such treatment may include eradication, or slowing of population growth, of a microbial agent associated with the inflammation.

By “prevention” we include the reduction in risk of coronavirus infection in patients. However, it will be appreciated that such prevention may not be absolute, i.e. it may not prevent all such patients developing a coronavirus infection, or may only partially prevent an infection in a single individual. As such, the terms “prevention” and “prophylaxis” may be used interchangeably.

In one embodiment, the coronavirus infection is an infection of the upper and/or lower respiratory tract. Alternatively, or additionally, the coronavirus infection may be in the gastrointestinal tract. Certain coronavirus, such as MERS, can also infect renal epithelial cells.

By “upper respiratory tract” we include the mouth, nose, sinus, middle ear, throat, larynx, and trachea.

By “lower respiratory tract” we include the bronchial tubes (bronchi) and the lungs (bronchi, bronchioles and alveoli), as well as the interstitial tissue of the lungs.

By “gastrointestinal tract” we mean the canal from the mouth to the anus, including the mouth, oesophagus, stomach and intestines.

In an alternative embodiment the coronavirus infection is a renal infection.

In one embodiment, the coronavirus infection is selected form the group consisting of common cold, pneumonia, pneumonitis, bronchitis, severe acute respiratory syndrome (SARS), Middle East respiratory syndrome (MERS), sinusitis, otitis and pharyngitis.

Other indications associated coronavirus infection are described in Gralinski & Baric, 2015, J. Pathol. 235:185-195 and Cavanagh, 2005, “Coronaviridae: a review of coronaviruses and toroviruses”, Coronaviruses with Special Emphasis on First Insights Concerning SARS 1, ed. by A. Schmidt, M. H. Wolff and O. Weber, Birkhäuser Verlag Basel, Switzerland (the disclosures of which are incorporated herein by reference).

Thus, in one preferred embodiment, the coronavirus infection is the common cold.

The coronavirus may be selected from the following:
(a) alphacoronavirus (such as human coronavirus 229E);
(b) betacoronavirus (such as human coronavirus HKU1);
(c) gammacoronavirus (such as avian coronavirus); and
(d) deltacoronavirus (such as bulbul coronavirus).

Examples of alphacoronaviruses include Alphacoronavirus 1, Bat coronavirus CDPHE15, Bat coronavirus HKU10, Human coronavirus 229E, Human coronavirus NL63, Miniopterus bat coronavirus 1, Miniopterus bat coronavirus HKU8, Mink coronavirus 1, Porcine epidemic diarrhoea virus, Rhinolophus bat coronavirus HKU2, and Scotophilus bat coronavirus 512.

Examples of betacoronaviruses include Murine coronavirus, Betacoronavirus 1, Hedgehog coronavirus 1, Human coronavirus HKU1, Middle East respiratory syndrome-related coronavirus, Pipistrellus bat coronavirus HKU5, Rousettus bat coronavirus HKU9, Severe acute respiratory syndrome-related coronavirus, and Tylonycteris bat coronavirus HKU4 Examples of gammacoronaviruses include Avian coronavirus and Beluga whale coronavirus SW1.

Examples of deltacoronaviruses include Bulbul coronavirus HKU11, Common moorhen coronavirus HKU21, Coronavirus HKU15, Munia coronavirus HKU13, Night heron coronavirus HKU19, Thrush coronavirus HKU12, White-eye coronavirus HKU16, and Wigeon coronavirus HKU20.

Thus, in an exemplary embodiment, the coronavirus is a human coronavirus.

For example, the coronavirus may be selected from:
(a) human coronavirus 229E;
(b) human coronavirus OC43;
(c) Severe Acute Respiratory Syndrome coronavirus (SARS-CoV)
(d) human Coronavirus NL63 (HCoV-NL63, New Haven coronavirus);
(e) human coronavirus HKU1; and
(f) Middle East respiratory syndrome coronavirus (MERS-CoV)

Accordingly, in one embodiment, the subject to be treated is human.

In one embodiment, the polypeptide having protease activity is selected from the group consisting of serine proteases, threonine proteases, cysteine proteases, aspartate proteases, glutamic acid proteases and metalloproteases.

In one embodiment, the polypeptide having protease activity is a serine protease. By “serine protease” we include both naturally occurring and non-naturally occurring catalytic polypeptides capable of cleaving peptide bonds in proteins, in which serine serves as the nucleophilic amino acid at the active site of the polypeptide (as defined in accordance with EC Number 3.4.21). The serine protease may have chymotrypsin-like protease activity (i.e. trypsins, chymotrypsins and elastases) or subtilisin-like protease activity.

Thus, in one embodiment the protease is a trypsin or chymotrypsin, or a component of a mixture thereof.

Thus, the polypeptides of the invention may exhibit trypsin activity. By “trypsin activity” we mean that the polypeptide exhibits a peptidase activity of a trypsin enzyme (EC 3,4,21,4) or of a related peptidase (such as chymotrypsin enzymes, EC 3,4,21,1). For example, the protease may be a naturally-occurring trypsin, of either eukaryotic or prokaryotic origin, or a mutated version of such a trypsin.

The polypeptides of the invention may be naturally occurring or non-naturally occurring.

In one embodiment, the polypeptide having protease activity comprises or consists of the amino acid sequence of a naturally-occurring protease (such as from marine, e.g. cod, or mammalian, e.g. bovine, sources). For example, the polypeptide having protease activity may consist of the amino acid sequence of a naturally-occurring trypsin, of either eukaryotic or prokaryotic origin.

In one embodiment, the polypeptide having protease activity is cold-adapted, i.e. the polypeptide is psychrophilic. By “cold-adapted” we mean the polypeptide is derived from an organism from a cold environment, and is hence adapted to function at low temperatures. For example, the polypeptide having protease activity may exhibit protease activity for longer periods of time at 15° C. than at higher temperatures, such as 25° C. or 37° C. (see Stefansson et al., 2010, Comparative Biochem. Physiol: Part B—Biochem. & Mol. Biol., 155(2): 186-194, the disclosures of which are incorporated by reference).

In a preferred embodiment, the polypeptide is a marine serine protease. The marine serine protease may be obtainable from, for example, cod, pollock, salmon or krill. Other possible sources of marine proteases include catfish, haddock, hoki, hake, redfish, roughies, tilapia, whiting and Chilean seabass. Specifically included are cold-adapted trypsins, such as a trypsin from Atlantic cod (Gadus morhua), Atlantic and Pacific salmon (e.g. Salmo salar and species of Oncorhynchus) and Alaskan Pollock (Theragra chalcogramma). For example, the polypeptide having serine protease activity may comprise or consist of the amino acid of any one of SEQ ID NOs:1 to 12, as listed below.

Thus, in a preferred embodiment, the marine serine protease is obtainable from Atlantic cod.

Naturally-occurring serine proteases may be purified or isolated from a source organism (e.g. Atlantic cod) or may be expressed recombinantly.

Thus, it will be appreciated by persons skilled in the art that such naturally-occurring serine protease polypeptides of the invention are provided in a form different to that in which they are found in nature. For example, the polypeptide of the invention may be isolated, in whole or in part, from the composition or form in which is it found in nature. Alternatively, the polypeptide of the invention may consist of the amino acid sequence of a naturally-occurring eukaryotic trypsin but expressed by recombinant means such that it has altered post-translational modification (e.g. glycosylation) compared to the protein as it is expressed in nature.

In a preferred embodiment, the marine serine protease is a trypsin, for example trypsin I, trypsin X, trypsin Y or trypsin ZT (for example, see below).

Three major isozymes of trypsin were originally characterised from Atlantic cod, designated Trypsin I, II and III (see Ásgeirsson et al., 1989, Eur. J. Biochem. 180:85-94, the disclosures of which are incorporated herein by reference). For example, trypsin I from Atlantic cod is defined in GenBank Accession No. AC090397 (see Stefansson et al, 2010, Comp. Biochem. Physiol. B, Biochem. Moi. Biol. 155 (2), 186-194, the disclosures of which are incorporated herein by reference). Subsequently, the trypsins produced by Atlantic cod have been further characterised and a number of distinct isoforms have now been characterised, including trypsin I, trypsin ZT, trypsin X and trypsin Y (see below).

In addition, Atlantic cod expresses two major isozymes of chymotrypsin, designated Chymotrypsin A and B (see Ásgeirsson & Bjarnason, 1991, Comp. Biochem. Physiol. B 998:327-335, the disclosures of which are incorporated herein by reference). For example, see GenBank Accession No. CAA55242.1.

In one embodiment, the polypeptide having protease activity comprises or consists of an amino acid sequence of trypsin I from Atlantic cod (Gadus morhua), i.e. SEQ ID NO: 1 or SEQ ID NO: 2

[SEQ ID NO: 1]
IVGGYECTKHSQAHQVSLNSGYHFCGGSLVSKDWVVSAAHCYKSRIEVRL
GEHHIRVNEGTEQYISSSSVIRHPNYSSYNINNDIMLIKLSKPATLNQYV
QPVALPTECAADGTMCTVSGWGNTMSSVADGDKLQCLSLPILSHADCANS
YPGMITQSMFCAGYLEGGKDSCQGDSGGPVVCNGVLQGVVSWGYGCAERD
HPGVYAKVCVLSGWVRDTMANY
[SEQ ID NO: 2]
IVGGYECTKHSQAHQVSLNSGYHFCGGSLVSKDWVVSAAHCYKSVLRVRL
GEHHIRVNEGTEQYISSSSVIRHPNYSSYNINNDIMLIKLTKPATLNQYV
HAVALPTECAADATMCTVSGWGNTMSSVADGDKLQCLSLPILSHADCANS
YPGMITQSMECAGYLEGGKDSCQGDSGGPVVCNGVLQGVVSWGYGCAERD
HPGVYAKVCVLSGWVRDTMANY

B 998:327-335, the disclosures of which are incorporated herein by reference.

or a fragment, variant, derivative or fusion thereof (or a fusion of said fragment, variant or derivative) of SEQ ID NO: 1 or 2, which retains the trypsin activity of said amino acid sequences.

Further details of trypsin I can be found in (see Guõmundsdóttir et al., 1993, Eur J Biochem. 217(3):1091-7 and Stefansson et al., 2010, Comp. Biochem. Physiol. B, Biochem. Moi. Biol. 155 (2), 186-194, (the disclosures of which are incorporated herein by reference).

Alternatively, the polypeptide having protease activity may comprise or consist of an amino acid sequence of a trypsin ZT isoform from Atlantic cod (Gadus morhua), e.g. SEQ ID NOs: 3 to 7 (see WO 2017/017012 to Enzymatica AB, the disclosures of which are incorporated herein by reference).

SEQ ID NO: 3 is the consensus sequence of the ZT-isoforms, ZT-1 to ZT-4, presented below.

[SEQ ID NO: 3]
IX₁GGX₂X₃CEPX₄SRPFMASLNYGYHFCGGVLINDQWVLSVAHOWYNPY
YMQVMLGEHDLRVFEGTEOLVKTNTIFWHEX₅YDYQTLDYDMMMIKLYHP
VEVTQSVAPISLPTGPPDGGMLCSVSGWGNMANGEEVNLPTRLQCLDVPI
VEX₆VX₇CX₈AX₉YPGMISPRMX₁₀CX₁₁GX₁₂MDGGRDX₁₃CNGDSGSP
LVCEGVLTGLVSWGX₁₄GCAX₁₅PNX₁₆PGVYVKVYEX₁₇LSWIQTTLD
ANP

wherein
X₁ is selected from I and V;
X₂ is selected from Q and H;
X₃ is selected from D and E;
X₄ is selected from R and N;
X₅ is L;
X₆ is selected from T and P;
X₇ is selected from D and A;
X₈ is selected from E and Q;
X₉ is selected from A and S;
X₁₀ is selected from V and M;
X₁₁ is selected from A and V;
X₁₂ is selected from Y and F;
X₁₃ is selected from A and V;
X₁₄ is selected from Q and R;
X₁₅ is selected from L and E;
X₁₆ is selected from Y and S; and
X₁₇ is selected from Y and F.

Atlantic cod trypsin ZT-1 isoform:
[SEQ ID NO: 4]
IVGGHECEPNSRPFMASLNYGYHFCGGVLINDQWVLSVAHCWYNPYYMQV
MLGEHDLRVFEGTEQLVKTNTIFWHELYDYQTLDYDMMMIKLYHPVEVTQ
SVAPISLPTGPPDGGMLCSVSGWGNMAMGEEVNLPTRLQCLDVPIVEPVA
CQASYPGMISPRMMCVGFMDGGRDVCNGDSGSPLVCEGVLTGLVSWGRGC
AEPNSPGVYVKVYEFLSWIQTTLDANP
Atlantic cod trypsin ZT-2 isoform:
[SEQ ID NO: 5]
IVGGHECEPNSRPFMASLNYGYHFCGGVLINDQWVLSVAHCWYNPYYMQV
MLGEHDLRVFEGTEQLVKTNTIFWHELYDYQTLDYDMMMIKLYHPVEVTQ
SVAPISLPTGPPDGGMLCSVSGWGNMAMGEEVNLPTRLQCLDVPIVETVD
CEAAYPGMISPRMVCAGYMDGGRDACNGDSGSPLVCEGVLTGLVSWGQGC
ALPNYPGVYVKVYEYLSWIQTTLDANP
Atlantic cod trypsin ZT-3 isoform:
[SEQ ID NO: 6]
IIGGQDCEPRSRPFMASLNYGYHFCGGVLINDQWVLSVAHCWYNPYYMQV
MLGEHDLRVFEGTEQLVKTNTIFWHELYDYQTLDYDMMMIKLYHPVEVTQ
SVAPISLPTGPPDGGMLCSVSGWGNMAMGEEVNLPTRLQCLDVPIVEPVA
CQASYPGMISPRMMCVGFMDGGRDVCNGDSGSPLVCEGVLTGLVSWGRGC
AEPNSPGVYVKVYEFLSWIQTTLDANP
Atlantic cod trypsin ZT-4 isoform:
[SEQ ID NO: 7]
IIGGQDCEPRSRPFMASLNYGYHFCGGVLINDQWVLSVAHCWYNPYYMQV
MLGEHDLRVFEGTEQLVKTNTIFWHELYDYQTLDYDMMMIKLYHPVEVTQ
SVAPISLPTGPPDGGMLCSVSGWGNMAMGEEVNLPTRLQCLDVPIVETVD
CEAAYPGMISPRMVCAGYMDGGRDACNGDSGSPLVCEGVLTGLVSWGQGC
ALPNYPGVYVKVYEYLSWIQTTLDANP

It will be appreciated by persons skilled in the art that the polypeptide may be present as a mixture of one or more of the above trypsin ZT isoforms, optionally in combination with trypsins I, X and/or Y.

Alternatively, the polypeptide having protease activity may comprise or consist of an amino acid sequence of trypsin X from Atlantic cod, e.g. SEQ ID NOs: 8 to 11 (see Stefansson et al., 2017, Biochim Biophys Acta. 1865(1):11-19, the disclosures of which are incorporated herein by reference).

Atlantic cod trypsin X:
[SEQ ID NO: 8]
IVGGYECTRHSQAHQVSLNSGYHFCGGSLVSKDWVVSAAHCYKSVLRVRL
GEHHIRVNEGTEQFISSSSVIRHPNYSSYNIDNDIMLIKLTEPATLNQYV
HAVALPTECAADATMCTVSGWGNTMSSVDDGDKLQCLNLPILSHADCANS
YPGMITQSMFCAGYLEGGKDSCQGDSGGPVVCNGVLQGVVSWGYGCAERD
NPGVYAKVCVLSGWVRDTMASY
Atlantic cod trypsin X-1:
[SEQ ID NO: 9]
IVGGYECTRHSQAHQVSLNSGYHFCGGSLVSKDWVVSAAHCYKSRIEVRL
GEHHIRVNEGTEQFISSSSVIRHPNYSSYNIDNDIMLIKLSEPATLNQYV
QPVALPTECAADGTMCTVSGWGNTMSSVDDGDKLQCLNLPILSHADCANS
YPGMITQSMFCAGYLEGGKDSCQGDSGGPVVCNGVLQGVVSWGYGCAERD
NPGVYAKVCVLSGWVRDTMASY
Atlantic cod trypsin X-2:
[SEQ ID NO: 10]
IVGGYECTRHSQAHQVSLNSGYHFCGGSLVSKDWVVSAAHCYKSRIEVRL
GEHHIRVNEGTEQFISSSSVIRHPNYSSYNIDNDIMLIKLSKPATLNQYV
QTVALPTECAADGTMCTVSGWGNTMSSVDDGDKLQCLNLPILSHADCSNS
YPGMITQSMFCAGYLEGGKDSCQGDSGGPVVCNGVLQGVVSWGYGCAERD
NPGVYAKVCVLSGWVRDTMASY
Atlantic cod trypsin X-3:
[SEQ ID NO: 11]
IVGGYECTRHSQAHQVSLNSGYHFCGGSLVSKDWVVSAAHCYKSRIEVRL
GEHHIRVNEGTEQFISSSSVIRHPNYSSYNIDNDIMLIKLSEPATLNQYV
QTVALPTECAADGTMCTVSGWGNTMSSVDDGDKLQCLNLPILSHADCSNS
YPGMITQSMFCAGYLEGGKDSCQGDSGGPVVCNGVLQGVVSWGYGCAERD
NPGVYAKVCVLSGWVRDTMASY

Alternatively, the polypeptide having protease activity may comprise or consist of an amino acid sequence of trypsin Y from Atlantic cod, e.g. SEQ ID NO: 12 (see Pálsdóttir & Gudmundsdóttir, 2008, Food Chem. 111(2):408-14, the disclosures of which are incorporated herein by reference).

Atlantic cod trypsin Y:
[SEQ ID NO: 12]
IIGGQDCEPRSRPFMASLNYGYHFCGGVLINDQWVLSVAHCWYNPYYMQV
MLGEHDLRVFEGTEQLVKTNTIFWHEQYDYQTLDYDMMMIKLYHPVEVTQ
SVAPISLPTGPPDGGMLCSVSGWGNMAMGEEVNLPTRLQCLDVPIVETVD
CEAAYPGMISPRMVCAGYMDGGRDACNGDSGSPLVCEGVLTGLVSWGQGC
ALPNYPGVYVKVYEYLSWIQTTLDANP

Thus, in exemplary embodiments, the polypeptide having protease activity comprises or consists of an amino acid sequence according to any one of SEQ ID NOs: 1 to 12. Such a polypeptide may be purified from Atlantic cod, for example as described in Ásgeirsson et al., 1989, Eur. J. Biochem. 180:85-94 (the disclosures of which are incorporated herein by reference).

Suitable exemplary polypeptides of the invention, and methods for their production, are also described in European Patent No. 1 202 743 B (the disclosures of which are incorporated herein by reference).

Like many proteases, trypsin I from Atlantic cod is produced as an inactive precursor, or zymogen, comprising a propeptide (or “activation”) sequence that is cleaved off to generate the mature, active trypsin. The initial expression product for trypsin also comprises a signal sequence, which is removed following expression.

The zymogen sequence for the variant of trypsin I from Atlantic cod corresponding to SEQ ID NO: 1, including the signal sequence, is shown below as SEQ ID NO: 13 (and corresponds to Genbank database accession no. ACO90397.1):

[SEQ ID NO: 13]
MKSLIEVLLLGAV [symbol] IVGGYECTKHSQAHQVSLNSGYHFCGGSLVS
KDWVVSAAHCYKSRIEVRLGEHHIRVNEGTEQYISSSSVIRHPNYSSYNI
NNDIMLIKLSKPATLNQYVQPVALPTECAADGTMCTVSGWGNTMSSVADG
DKLQCLSLPILSHADCANSYPGMITQSMECAGYLEGGKDSCQGDSGGPVV
CNGVLQGVVSWGYGCAERDHPGVYAKVCVLSGWVRDTMANY

wherein:
Signal peptide=amino acids 1 to 13 (underlined)
Propeptide=amino acids 14 to 19 (bold italics)
Mature trypsin=amino acids 20 to 241

The zymogen sequence for the variant trypsin I from Atlantic cod corresponding to SEQ ID NO: 2, including the signal sequence, is shown below as SEQ ID NO: 14 (and corresponds to Uniprot database accession no. P16049-1):

```
[SEQ ID NO: 14]
                10 20 30 40
MKSLIFVLLL GAV [symbol] I VGGYECTKHS QAHQVSLNSG
                50 60 70 80
YHFCGGSLVS KDWVVSAAHC YKSVLRVRLG EHHIRVNEGT
                90 100 110 120
EQYISSSSVI RHPNYSSYNI NNDIMLIKLT KPATLNQYVH
              130 140 150 160
AVALPTECAA DATMCTVSGW GNTMSSVADG DKLQCLSLPI
              170 180 190 200
LSHADCANSY PGMITQSMFC AGYLEGGKDS CQGDSGGPVV
              210 220 230 240
CNGVLQGVVS WGYGCAERDH PGVYAKVCVL SGWVRDTMAN Y
```

wherein:
Signal peptide=amino acids 1 to 13 (underlined)
Propeptide=amino acids 14 to 19 (bold italics)
Mature trypsin=amino acids 20 to 241

The zymogen sequence for the variant trypsin X corresponding to SEQ ID NO: 8, including the signal sequence, is shown below as SED ID NO: 15 (and corresponds to Genbank Accession No. Q91041.2)

[SEQ ID NO: 15]
MKSLIFVLLLGAV [symbol] IVGGYECTRHSQAHQVSLNSGYHFCGGSLVS
KDWVVSAAHCYKSVLRVRLGEHHIRVNEGTEQFISSSSVIRHPNYSSYNI
DNDIMLIKLTEPATLNQYVHAVALPTECAADATMCTVSGWGNTMSSVDDG
DKLQCLNLPILSHADCANSYPGMITQSMFCAGYLEGGKDSCQGDSGGPVV
CNGVLQGVVSWGYGCAERDNPGVYAKVCVLSGWVRDTMASY

(wherein the signal sequence and propeptide are underlined and in bold italics, respectively).

The zymogen sequence for the variant trypsin X-1 corresponding to SEQ ID NO: 9, including the signal sequence, is shown below as SED ID NO: 16 (and corresponds to Genbank Accession No. AOX15769.1)

[SEQ ID NO: 16]
MKSLIFVLLLGAV [symbol] IVGGYECTRHSQAHQVSLNSGYHFCGGSLVS
KDWVVSAAHCYKSRIEVRLGEHHIRVNEGTEQFISSSSVIRHPNYSSYNI
DNDIMLIKLSEPATLNQYVQPVALPTECAADGTMCTVSGWGNTMSSVDDG
DKLQCLNLPILSHADCANSYPGMITQSMFCAGYLEGGKDSCQGDSGGPVV
CNGVLQGVVSWGYGCAERDNPGVYAKVCVLSGWVRDTMASY

(wherein the signal sequence and propeptide are underlined and in bold italics, respectively).

The zymogen sequence for the variant trypsin X-2 corresponding to SEQ ID NO: 10, including the signal sequence, is shown below as SED ID NO: 17 (and corresponds to Genbank Accession No. AOX15770.1)

[SEQ ID NO: 17]
MKSLIFVLLLGAV [symbol] IVGGYECTRHSQAHQVSLNSGYHFCGGSLVS
KDWVVSAAHCYKSRIEVRLGEHHIRVNEGTEQFISSSSVIRHPNYSSYNI
DNDIMLIKLSKPATLNQYVQTVALPTECAADGTMCTVSGWGNTMSSVDDG
DKLQCLNLPILSHADCSNSYPGMITQSMFCAGYLEGGKDSCQGDSGGPVV
CNGVLQGVVSWGYGCAERDNPGVYAKVCVLSGWVRDTMASY

(wherein the signal sequence and propeptide are underlined and in bold italics, respectively).

The zymogen sequence for the variant trypsin X-3 corresponding to SEQ ID NO: 11, including the signal sequence, is shown below as SED ID NO: 18 (and corresponds to Genbank Accession No. AOX15771.1)

[SEQ ID NO: 18]
MKSLIFVLLLGAV [symbol] IVGGYECTRHSQAHQVSLNSGYHFCGGSLVS
KDWVVSAAHCYKSRIEVRLGEHHIRVNEGTEQFISSSSVIRHPNYSSYNI
DNDIMLIKLSEPATLNQYVQTVALPTECAADGTMCTVSGWGNTMSSVDDG
DKLQCLNLPILSHADCSNSYPGMITQSMFCAGYLEGGKDSCQGDSGGPVV
CNGVLQGVVSWGYGCAERDNPGVYAKVCVLSGWVRDTMASY

(wherein the signal sequence and propeptide are underlined and in bold italics, respectively).

The zymogen sequence for the variant trypsin Y corresponding to SEQ ID NO: 12, including the signal sequence, is shown below as SED ID NO: 19 (and corresponds to Genbank Accession No. CAD30563.1)

[SEQ ID NO: 19]
MIGLALLMLLGAAAAV [symbol] IIGGQDCEPRSRPFMASLNYGYHFCGGV
LINDQWVLSVAHCWYNPYYMQVMLGEHDLRVFEGTEQLVKTNTIFWHEQY
DYQTLDYDMMMIKLYHPVEVTQSVAPISLPTGPPDGGMLCSVSGWGNMAM
GEEVNLPTRLQCLDVPIVETVDCEAAYPGMISPRMVCAGYMDGGRDACNG
DSGSPLVCEGVLTGLVSWGQGCALPNYPGVYVKVYEYLSWIQTTLDANP

(wherein the signal sequence and propeptide are underlined and in bold italics, respectively).

The trypsin ZT isoforms represented by SEQ ID NOs: 3 to 7 represent the active variants of these trypsins, i.e. variants that have been activated by cleavage of the N terminus of the trypsins. These trypsins are proteins expressed in the pyloric caeca/pancreas (pancreatic tissue in fish) with a number of amino acids on the N terminal end that are important for secretion out of the cells and for keeping the enzyme inactive.

For example, the full-length trypsin ZT isoforms are also disclosed herein as:

Uncleaved Atlantic cod trypsin ZT-1 isoform:
[SEQ ID NO: 20]
MIGLALLMLLGAAAAAVPRDVGKIVGGHECEPNSRPFMASLNYGYHFCGG
VLINDQWVLSVAHCWYNPYYMQVMLGEHDLRVFEGTEQLVKTNTIFWHEL
YDYQTLDYDMMMIKLYHPVEVTQSVAPISLPTGPPDGGMLCSVSGWGNMA
MGEEVNLPTRLQCLDVPIVEPVACQASYPGMISPRMMCVGFMDGGRDVCN
GDSGSPLVCEGVLTGLVSWGRGCAEPNSPGVYVKVYEFLSWIQTTLDANP
Uncleaved Atlantic cod trypsin ZT-2 isoform:
[SEQ ID NO: 21]
MIGLALLMLLGAAAAAVPRDVGKIVGGHECEPNSRPFMASLNYGYHFCGG
VLINDQWVLSVAHCWYNPYYMQVMLGEHDLRVFEGTEQLVKTNTIFWHEL
YDYQTLDYDMMMIKLYHPVEVTQSVAPISLPTGPPDGGMLCSVSGWGNMA
MGEEVNLPTRLQCLDVPIVETVDCEAAYPGMISPRMVCAGYMDGGRDACN
GDSGSPLVCEGVLTGLVSWGQGCALPNYPGVYVKVYEYLSWIQTTLDANP
Uncleaved Atlantic cod trypsin ZT-3 isoform:
[SEQ ID NO: 22]
MIGLALLMLLGAAAAVPREDGRIIGGQDCEPRSRPFMASLNYGYHFCGGV
LINDQWVLSVAHCWYNPYYMQVMLGEHDLRVFEGTEQLVKTNTIFWHELY
DYQTLDYDMMMIKLYHPVEVTQSVAPISLPTGPPDGGMLCSVSGWGNMAM
GEEVNLPTRLQCLDVPIVEPVACQASYPGMISPRMMCVGFMDGGRDVCNG
DSGSPLVCEGVLTGLVSWGRGCAEPNSPGVYVKVYEFLSWIQTTLDANP
Uncleaved Atlantic cod trypsin ZT-4 isoform:
[SEQ ID NO: 23]
MIGLALLMLLGAAAAVPREDGRIIGGQDCEPRSRPFMASLNYGYHFCGGV
LINDQWVLSVAHCWYNPYYMQVMLGEHDLRVFEGTEQLVKTNTIFWHELY
DYQTLDYDMMMIKLYHPVEVTQSVAPISLPTGPPDGGMLCSVSGWGNMAM
GEEVNLPTRLQCLDVPIVETVDCEAAYPGMISPRMVCAGYMDGGRDACNG
DSGSPLVCEGVLTGLVSWGQGCALPNYPGVYVKVYEYLSWIQTTLDANP

The term ‘amino acid’ as used herein includes the standard twenty genetically-encoded amino acids and their corresponding stereoisomers in the ‘D’ form (as compared to the natural ‘L’ form), omega-amino acids and other naturally-occurring amino acids, unconventional amino acids (e.g., α,α-disubstituted amino acids, N-alkyl amino acids, etc.) and chemically derivatised amino acids (see below).

When an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

In accordance with convention, the amino acid sequences disclosed herein are provided in the N-terminus to C-terminus direction.

Typically, the polypeptides of the invention comprise or consist of L-amino acids.

Persons of skill in the art will appreciate that the polypeptide having protease activity may comprise or consist of a fragment, variant, derivative or fusion thereof (or a fusion of said fragment, variant or derivative) of one of the above amino acid sequences, e.g. SEQ ID NOs: 1 to 12, provided that said fragment, variant, derivative or fusion retains (at least in part) the trypsin activity of said amino acid sequences.

Trypsin activity may be determined using methods well known in the art. For example, trypsin assay kits are commercially available from Abcam, Cambridge, UK (see Cat No. ab102531) and other suppliers. In one embodiment, trypsin activity is measured using Cbz-Gly-Pro-Arg-p-nitroanilide (Cbz-GPR-pNA) as a substrate (see EP 1,202,743 B and Stefansson et al, 2010, Comp Biochem Physiol B Biochem Mol Biol. 155(2):186-94, the disclosures of which are incorporated herein by reference).

Typically, the protease polypeptide has a specific activity of at least 1 U/mg of polypeptide, for example at least 10 U/mg, at least 50 U/mg, at least 100 U/mg, at least 200 U/mg or at least 500 U/mg. ‘U’ as used herein means an enzyme unit (one U is the amount of enzyme that catalyzes the conversion of 1 micro-mole of substrate per minute).

Thus, where the polypeptide comprises an amino acid sequence according to any one of SEQ ID NOs: 1 to 12, it may comprise additional amino acids at its N- and/or C-terminus beyond those of SEQ ID NOs: 1 to 12. Likewise, where the polypeptide comprises a fragment, variant or derivative of an amino acid sequence according to SEQ ID NOs: 1 to 12, it may comprise additional amino acids at its N- and/or C-terminus.

Alternatively, the polypeptide having protease activity may correspond to a fragment of such a wildtype trypsin, such as SEQ ID NOs: 1 to 12, provided that said fragment retains (at least in part) the trypsin activity of the naturally occurring trypsin protein from which it is derived. Thus, the polypeptide may comprise or consist of at least 10 contiguous amino acids of SEQ ID NOs: 1 to 12, e.g. at least 15, 16, 17, 18, 19, 20, 30, 40, 50, 60, 70, 80, 90, 100, 110, 120, 130, 140, 150, 160, 170, 180, 190, 200, 210, 220, 230 or 240 contiguous amino acids any one of SEQ ID NOs: 1 to 12.

For example, the fragment may comprise or consist of amino acid residues 61 to 77 of any one of SEQ ID NOs:1 to 12. Alternatively, or in addition, the fragment may comprise or consist of amino acid residues 225 to 241 of any one of SEQ ID NOs: 1 to 12.

It will be appreciated by persons skilled in the art that the polypeptide of the invention may alternatively comprise or consist of a variant of the amino acid sequence according to any one of SEQ D NOs: 1 to 12 (or fragments thereof). Such a variant may be a non-naturally occurring variant.

By ‘variants’ of the polypeptide we include insertions, deletions and substitutions, either conservative or non-conservative. In particular, we include variants of the polypeptide where such changes retain, at least in part, the trypsin activity of the said polypeptide.

Such variants may be made using the methods of protein engineering and site-directed mutagenesis well known in the art using the recombinant polynucleotides (see Molecular Cloning: a Laboratory Manual, 4th edition, Green & Sambrook, 2012, Cold Spring Harbor Laboratory Press, which is incorporated herein by reference).

In one embodiment, the variant has an amino acid sequence which has at least 50% identity with the amino acid sequence according to any one of SEQ ID NOs: 1 to 12, or a fragment thereof, for example at least 55%, 60%, 65%, 70%, 75%, 80%, 90%, 95%, 96%, 97%, 98% or at least 99% identity.

The percent sequence identity between two polypeptides may be determined using suitable computer programs, for example the GAP program of the University of Wisconsin Genetic Computing Group and it will be appreciated that percent identity is calculated in relation to polypeptides whose sequences have been aligned optimally.

The alignment may alternatively be carried out using the Clustal W program (as described in Thompson et al., 1994, Nuc. Acid Res. 22:4673-4680, which is incorporated herein by reference).

The parameters used may be as follows:
Fast pairwise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

Examples of variant polypeptides having protease activity are disclosed in WO 2015/150799 to Enzymatica AB, the disclosures of which are incorporated by reference.

In a further embodiment of the first aspect of the invention, the polypeptide having protease activity comprises or consists of a fusion protein.

By ‘fusion’ of a polypeptide we include an amino acid sequence corresponding to a polypeptide having protease activity (such as SEQ ID NOS:1 to 12 or a fragment or variant thereof) fused to any other polypeptide. For example, the said polypeptide may be fused to a polypeptide such as glutathione-S-transferase (GST) or protein A in order to facilitate purification of said polypeptide. Examples of such fusions are well known to those skilled in the art. Similarly, the said polypeptide may be fused to an oligo-histidine tag such as His6 or to an epitope recognised by an antibody such as the well-known Myc tag epitope. Fusions to any variant or derivative of said polypeptide are also included in the scope of the invention.

The fusion may comprise a further portion which confers a desirable feature on the said polypeptide of the invention; for example, the portion may be useful in augmenting or prolonging the therapeutic effect. For example, in one embodiment the fusion comprises human serum albumin or a similar protein.

Alternatively, the fused portion may be, for example, a biotin moiety, a radioactive moiety, a fluorescent moiety, for example a small fluorophore or a green fluorescent protein (GFP) fluorophore, as well known to those skilled in the art. The moiety may be an immunogenic tag, for example a Myc tag, as known to those skilled in the art or may be a lipophilic molecule or polypeptide domain that is capable of promoting cellular uptake of the polypeptide, as known to those skilled in the art.

In a further embodiment of the first aspect of the invention, the polypeptide, or fragment, variant, fusion or derivative thereof, comprises or consists of one or more amino acids that are modified or derivatised.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. Thus, by ‘polypeptide’ we include peptidomimetic compounds which have a protease activity of the polypeptide of any of SEQ ID NOS: 1 to 12. The term ‘peptidomimetic’ refers to a compound that mimics the conformation and desirable features of a particular peptide as a therapeutic agent.

For example, the polypeptides of the invention include not only molecules in which amino acid residues are joined by peptide (—CO—NH—) linkages but also molecules in which the peptide bond is reversed. Such retro-inverso peptidomimetics may be made using methods known in the art, for example such as those described in Meziere et a. (1997) J. Immunol 159, 3230-3237, which is incorporated herein by reference. This approach involves making pseudopeptides containing changes involving the backbone, and not the orientation of side chains. Retro-inverse peptides, which contain NH—CO bonds instead of CO—NH peptide bonds, are much more resistant to proteolysis. Alternatively, the polypeptide of the invention may be a peptidomimetic compound wherein one or more of the amino acid residues are linked by a -y(CH₂NH)— bond in place of the conventional amide linkage.

In a further alternative, the peptide bond may be dispensed with altogether provided that an appropriate linker moiety which retains the spacing between the carbon atoms of the amino acid residues is used; it may be advantageous for the linker moiety to have substantially the same charge distribution and substantially the same planarity as a peptide bond.

It will be appreciated that the polypeptide may conveniently be blocked at its N- or C-terminus so as to help reduce susceptibility to exoproteolytic digestion.

A variety of uncoded or modified amino acids such as d-amino acids and N-methyl amino acids have also been used to modify polypeptides. In addition, a presumed bioactive conformation may be stabilised by a covalent modification, such as cyclisation or by incorporation of lactam or other types of bridges, for example see Veber et al., 1978, Proc. Natl. Acad. Sci. USA 75:2636 and Thorsett et al., 1983, Biochem. Biophys. Res. Comm. 111:166, which are incorporated herein by reference.

In one preferred embodiment, however, the polypeptide of the invention comprises one or more amino acids modified or derivatised by PEGylation, amidation, esterification, acylation, acetylation and/or alkylation.

It will be appreciated by persons skilled in the art that the polypeptide having protease activity may be of any suitable length. In one embodiment, the polypeptides are between 10 and 30 amino acids in length, for example between 10 and 20, 12 and 18, 12 and 16, or 15 and 20 amino acids in length. Alternatively, the polypeptide may be between 150 and 250 amino acids in length, for example between 200 and 250, 210 and 240, 220 and 230, or 220 and 225 amino acids in length.

Typically, the polypeptide is linear.

Where the polypeptide having protease activity is a naturally occurring protease, the protein may be extracted and/or purified (e.g. isolated) from its natural source using methods known in the art.

For example, marine-derived trypsins such as trypsins from Atlantic cod may be produced using the extraction methods described in EP 1 202 743 B to Jon Bragi Bjarnason, the disclosures of which are incorporated by reference.

In an alternative embodiment, the polypeptide having protease activity may be produced by recombinant methods.

Thus, the polypeptides for use in the invention, as well as nucleic acid molecules, vectors and host cells for producing the same, may be made using methods well known in the art (for example, see Green & Sambrook, 2012, Molecular Cloning, A Laboratory Manual, Fourth Edition, Cold Spring Harbor, N.Y., the relevant disclosures in which document are hereby incorporated by reference).

Recombinant methods for producing polypeptides having protease activity, such as trypsins, are disclosed in WO 2015/150799 to Enzymatica, the disclosures of which are incorporated by reference.

In a further alternative embodiment, the polypeptides having protease activity may be synthesised by known means, such as liquid phase and solid phase synthesis (for example, t-Boc solid-phase peptide synthesis and BOP-SPPS).

It will be appreciated by persons skilled in the art that the present invention also includes the use of pharmaceutically acceptable acid or base addition salts of the above described polypeptides. The acids which are used to prepare the pharmaceutically acceptable acid addition salts of the aforementioned base compounds useful in this invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

Pharmaceutically acceptable base addition salts may also be used to produce pharmaceutically acceptable salt forms of the polypeptides. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others.

It will be further appreciated that the polypeptides of the invention may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of activity loss and that use levels may have to be adjusted upward to compensate. Preferably, the lyophilised (freeze dried) polypeptide loses no more than about 20%, or no more than about 25%, or no more than about 30%, or no more than about 35%, or no more than about 40%, or no more than about 45%, or no more than about 50% of its activity (prior to lyophilisation) when rehydrated.

The polypeptides having protease activity are typically provided in the form of a therapeutic composition, in which the polypeptide is formulated together with a pharmaceutically acceptable buffer, diluent, carrier, adjuvant or excipient. Additional compounds may be included in the compositions, including, chelating agents such as EDTA, citrate, EGTA or glutathione. The antimicrobial/therapeutic compositions may be prepared in a manner known in the art that is sufficiently storage stable and suitable for administration to humans and animals. The therapeutic compositions may be lyophilised, e.g., through freeze drying, spray drying, spray cooling, or through use of particle formation from supercritical particle formation.

It will be appreciated by persons skilled in the art that the polypeptides of the invention may also be added to formulations for topical application, e.g. as a cosmetic composition, a pharmaceutical composition or a medical device, in order to impart a therapeutic, prophylactic and/or cosmetic benefit to a product (such as eardrops, wash compositions and the like). The terms ‘pharmaceutical composition’ and ‘medicament’ as used herein are to be construed accordingly.

By “pharmaceutically acceptable” we mean a non-toxic material that does not decrease the effectiveness of the trypsin activity of the polypeptide of the invention. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), the disclosures of which are incorporated herein by reference).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the peptide in the therapeutic preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the polypeptide of the invention. The adjuvant may be one or more of zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and acetates of different acyl composition. The adjuvant may also be cationic polymers such as cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, glucose, sucrose, mannitol, and cyclodextrines, which are added to the composition, e.g., for facilitating lyophilisation. Examples of polymers are starch, cellulose ethers, cellulose carboxymethycelluose, hydroxypropylmethyl cellulose, hydroxyethyl cellulose, ethylhydroxyethyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

In one embodiment, the polypeptide may be provided together with a stabiliser, such as calcium chloride.

The polypeptides of the invention may be formulated into any type of therapeutic composition known in the art to be suitable for the delivery of polypeptide agents.

In one embodiment, the polypeptides may simply be dissolved in water, saline, polyethylene glycol, propylene glycol, ethanol or oils (such as safflower oil, corn oil, peanut oil, cottonseed oil or sesame oil), tragacanth gum, and/or various buffers.

In one embodiment, the invention provides a protease polypeptide as described above in an osmotically active solution. For example, the polypeptide may be formulated in glycerol or glycerine.

In a further embodiment, the therapeutic compositions of the invention may be in the form of a liposome, in which the polypeptide is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations is can be found in for example U.S. Pat. No. 4,235,871, the disclosures of which are incorporated herein by reference.

The therapeutic compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microspheres. Preparations of such microspheres can be found in U.S. Pat. No. 5,851,451 and in EP 0 213 303, the disclosures of which are incorporated herein by reference.

In a further embodiment, the therapeutic compositions of the invention are provided in the form of polymer gels, where polymers such as starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polyvinyl imidazole, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone are used for thickening of the solution containing the peptide. The polymers may also comprise gelatin or collagen.

It will be appreciated that the therapeutic compositions of the invention may include ions and a defined pH for potentiation of action of the polypeptides. Additionally, the compositions may be subjected to conventional therapeutic operations such as sterilisation and/or may contain conventional adjuvants such as preservatives, stabilisers, wetting agents, emulsifiers, buffers, fillers, etc.

In one preferred embodiment, the therapeutic composition comprises the polypeptide in a Tris or phosphate buffer, together with one or more of EDTA, xylitol, sorbitol, propylene glycol and glycerol.

In one embodiment the polypeptide is for administration in combination with glycerol and a buffer.

The therapeutic compositions according to the invention may be administered via any suitable route known to those skilled in the art. Thus, possible routes of administration include oral, buccal, parenteral (intravenous, subcutaneous, and intramuscular), topical, ocular, nasal, pulmonar, parenteral, vaginal and rectal. Also, administration from implants is possible. The polypeptide may be formulated as a spray, gel, cream or liquid or conventional liquid for administration.

In one preferred embodiment, the therapeutic compositions are administered topically, in a form suitable for delivery to the oropharynx. For example, the polypeptide may be formulated as a mouth spray, lozenge, pastille, tablet, syrup or chewing gum.

In an alternative embodiment, the therapeutic compositions are administered parentally, for example, intravenously, intracerebroventricularly, intraarticularly, intra-arterially, intraperitoneally, intrathecally, intraventricularly, intrasternally, intracranially, intramuscularly or subcutaneously, or they may be administered by infusion techniques. They are conveniently used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

The therapeutic compositions will be administered to a patient in a pharmaceutically effective dose. A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art. The administration of the pharmaceutically effective dose can be carried out both by single administration in the form of an individual dose unit or else several smaller dose units and also by multiple administrations of subdivided doses at specific intervals. Alternatively, the dose may be provided as a continuous infusion over a prolonged period.

The protease polypeptides can be formulated at various concentrations, depending on the efficacy/toxicity of the polypeptide being used.

In one embodiment, the formulation comprises the protease polypeptide at a concentration of between 0.01 and 100 U/g of the formulation, e.g. between 1 and 10 U/g of the formulation. For example, the formulation (e.g. mouthwash, gel, ointment, etc.) may comprise at least 0.1 U/g, at least 0.5 U/g, at least 1 U/g, at least 5 U/g, at least 10 U/g, or at least 50 U/g of the protease polypeptide in the formulation. Thus, the formulation (e.g. mouthwash, gel, ointment, etc.) may comprise no more than 50 U/g, no more than 20 U/g, no more than 10 U/g, no more than 5 U/g, no more than 1 U/g, or no more than 0.1 U/g of the protease polypeptide in the formulation.

Thus, the therapeutic formulation may comprise an amount of a polypeptide, or fragment, variant, fusion or derivative thereof, sufficient to kill or slow the growth of microorganisms, such as viruses, bacteria and yeasts, within the ear.

Where the polypeptide is formulated for administration to the oropharynx (for example as a spray or gel), the therapeutic composition may comprise the polypeptide dissolved in water and glycerol. Exemplary mouth spray and gel formulations have been marketed as Coldzyme® (by Enzymatica AB, Lund, Sweden) and PreCold® (by Zymetech ehf, Reykjavik, Iceland).

In one embodiment the polypeptide can be provided in a delivery device, for example in a spray container, which may be configured for ease of delivery to the oropharynx.

In one embodiment the polypeptide is for use in combination with one or more additional active agents.

For example, the additional active agents may be selected from the group consisting of antimicrobial agents (including antibiotics, antiviral agents and anti-fungal agents), anti-inflammatory agents (including steroids and non-steroidal anti-inflammatory agents) and antiseptic agents.

In one embodiment the active agents are one or more antimicrobial agents, for example antibiotics selected from the group consisting of penicillins, cephalosporins, fluoroquinolones, aminoglycosides, monobactams, carbapenems and macrolides.

For example, the antibiotics may be selected from the group consisting of amikacin, amoxicillin, ampicillin, azithromycin, carbenicillin, carbapenems, cefotaxime, ceftazidime, ceftriaxone, cefuroxime, cephalosporins, chloramphenicol, ciprofloxacin, clindamycin, dalacin, dalfopristin, daptomycin, doxycycline, enrofloxacin, ertapenem, erythromycin, fluoroquinolones, gentamicin, marbofloxacin, meropenem, metronidazole, minocycline, moxifloxacin, nafcillin, ofloxacin, oxacillin, penicillin, quinupristin, rifampin, silver sulfadiazine, sulfamethoxazole, teicoplanin, tetracycline, tobramycin, trimethoprim, vancomycin, bacitracin and polymyxin B, or a mixture thereof.

In one preferred embodiment, the one or more antibiotic compounds is/are selected from the group consisting of tetracycline, cefotaxime, vancomycin, erythromycin and oxacillin.

It will be appreciated by persons skilled in the art that the combinations therapies of the invention may comprise a single antibiotic compound or multiple antibiotic compounds.

The concentration of the antibiotics to be used in the combination therapies of the invention will depend on the particular antibiotic to be used and the indication and/or location of the biofilm to be treated, in accordance with common general knowledge in the field. Typically, the antibiotic will be formulated at a concentration of between 0.1 to 5% (by weight), for example between 0.1 to 1% (by weight).

In one embodiment, the additional antibiotics may be for topical or oral administration.

In one embodiment, the invention provides an implantable medical device which is impregnated, coated or otherwise treated with a polypeptide as described herein.

A second related aspect of the invention provides a polypeptide having protease activity for use in the preparation of a medicament for the treatment or prevention of coronavirus infection in a mammal.

Examples of suitable polypeptides having protease activity are detailed above in relation to the first aspect of the invention. In particular, the polypeptide may be a trypsin or chymotrypsin, or a component of a mixture thereof.

In one embodiment of the second aspect of the invention, the polypeptide comprises or consists of an amino acid sequence of any one of SEQ ID NOs: 1 to 12, or a fragment, variant, derivative or fusion thereof (or a fusion of said fragment, variant or derivative) which retains the trypsin activity of said amino acid sequence.

Thus, in one embodiment the polypeptide consists of an amino acid sequence of any one of SEQ ID NOs: 1 to 12.

In one embodiment, the coronavirus infection is selected form the group consisting of common cold, pneumonia, bronchitis, severe acute respiratory syndrome (SARS), Middle East respiratory syndrome (MERS), sinusitis, otitis and pharyngitis.

A third related aspect of the invention provides a method for treatment or prevention of coronavirus infection in a mammal comprising administering to the subject a therapeutically-effective amount of a polypeptide having protease activity.

Examples of suitable polypeptides having protease activity are detailed above in relation to the first aspect of the invention. In particular, the polypeptide may be a trypsin or chymotrypsin, or a component of a mixture thereof.

In one embodiment of the third aspect of the invention, the polypeptide comprises or consists of an amino acid sequence of any one of SEQ ID NOs: 1 to 12, or a fragment, variant, derivative or fusion thereof (or a fusion of said fragment, variant or derivative) which retains the trypsin activity of said amino acid sequence.

Thus, in one embodiment the polypeptide consists of an amino acid sequence of any one of SEQ ID NOs: 1 to 12.

In one embodiment, the coronavirus infection is selected form the group consisting of common cold, pneumonia, bronchitis, severe acute respiratory syndrome (SARS), Middle East respiratory syndrome (MERS), sinusitis, otitis and pharyngitis.

Preferences and options for a given aspect, feature or parameter of the invention should, unless the context indicates otherwise, be regarded as having been disclosed in combination with any and all preferences and options for all other aspects, features and parameters of the invention. For example, in one embodiment the invention provides a polypeptide consisting of an amino acid sequence of any one of SEQ ID NO: 1 to 7 for use in the treatment of a coronavirus infection in a human.

The listing or discussion of an apparently prior-published document in this specification should not necessarily be taken as an acknowledgement that the document is part of the state of the art or is common general knowledge.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the above description. It should be understood, however, that the above description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

Preferred, non-limiting examples which embody certain aspects of the invention will now be described.

The listing or discussion of an apparently prior-published document in this specification should not necessarily be taken as an acknowledgement that the document is part of the state of the art or is common general knowledge.

Preferred, non-limiting examples which embody certain aspects of the invention will now be described, with reference to the following figures:

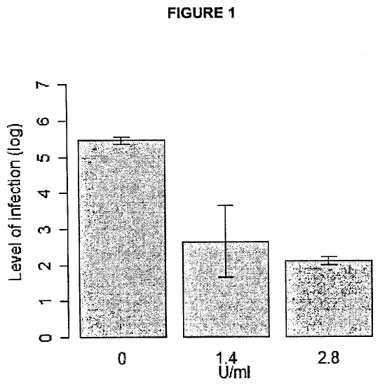

FIG. 1. Deactivation of coronavirus (CoV-229E-luc) by cod trypsin. This figure shows that cod trypsin lowers the ability of coronavirus in infecting human liver cells (Huh7). Y-bar shows the level of infection by the coronavirus on a log scale based on results from a Renilla luciferase assay (see Methods). X-bar shows the amount of cod trypsin used in U/mL.

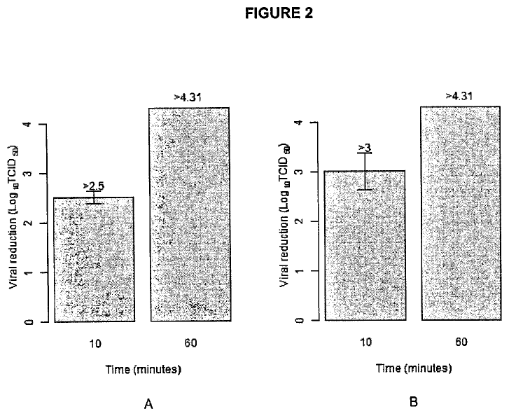

FIG. 2. Deactivation of coronavirus (CoV-229E) by cod trypsin. This figure shows that cod trypsin lowers the ability of coronavirus in infecting human fetal lung fibroblast-like cells (MRC5). Y-bar shows the reduction of infection by the coronavirus on a log scale. The concentration of cod trypsin used was 1.22 U/mL (panel A) and 2.44 U/mL (panel B). Each concentration and time point was done in duplicate.

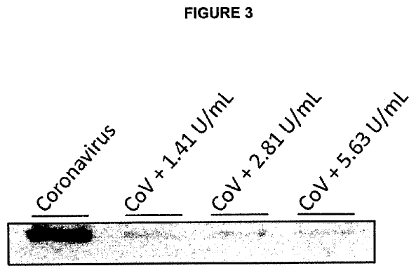

FIG. 3. Western blot analysis of coronavirus proteins in a coronavirus sample incubated with different concentrations of cod trypsin. This figure shows that coronavirus proteins are degraded in samples containing cod trypsin treated coronavirus. Proteins within cod trypsin treated coronavirus samples were resolved by SDS-PAGE and subjected to Western blot analyses using an antibody directed against coronaviral proteins (see Methods). The concentration of cod trypsin used is shown above each lane in the gel. The lane labelled coronavirus is a sample that did not contain cod trypsin.

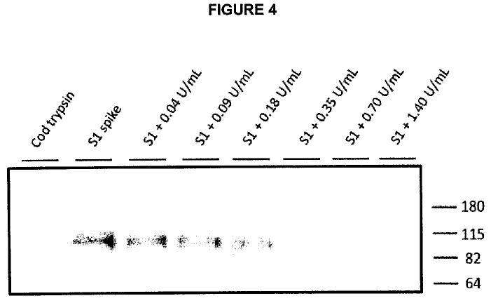

FIG. 4. Western blot analysis of recombinant CoV-229E spike protein incubated with different concentrations of cod trypsin. This figure shows that recombinant coronavirus spike protein is degraded by cod trypsin. Proteins within samples containing recombinant coronavirus spike protein incubated with different concentrations of cod trypsin were resolved by SDS-PAGE and subjected to Western blot analyses. Migration of size standards is shown by bars and numbers (kDa) on the right side. As controls, a sample containing cod trypsin and a sample containing S1 spike protein were loaded on the gel. The amount of cod trypsin incubated with recombinant coronavirus spike protein is shown above each lane in the gel.

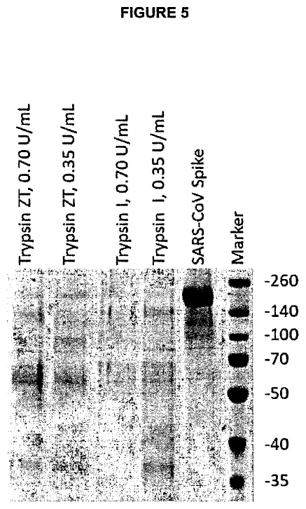

FIG. 5. SDS-PAGE analysis of recombinant SARS-CoV spike protein incubated with trypsin ZT or trypsin I for 10 min at 33° C. This figure shows that recombinant SARS-CoV spike protein is degraded by trypsin ZT and trypsin I. Proteins within samples containing recombinant SARS-CoV spike protein incubated with different concentrations of cod trypsin were resolved by SDS-PAGE. The gel was dyed with Coomassie blue and imaged on an Odyssey infrared imaging system. Migration of size standards is shown by bars and numbers (kDa) on the right side. As a control, a sample containing SARS-CoV spike protein was loaded on the gel. The amount of trypsin ZT and trypsin I incubated is shown above each lane in the gel.

### EXAMPLES

### Materials & Methods

### Cells, Viruses and Enzymes

Huh7 cells and CoV-229E-luc (van den Worm, Eriksson et al. 2012) were obtained from Prof. Dr. Volker Thiel at the Institute of Virology and Immunology, University of Bern, Switzerland. Benzamidine purified cod trypsin was obtained from Zymetech (Reykjavik, Iceland). Trypsin ZT (unpublished data in a manuscript submitted for publication: Sandholt G B, Stefansson B, Gudmundsdottir A) and trypsin I were purified as previously described (Stefansson, Helgadottir et al. 2010). Activity of cod trypsin was measured using the substrate CBZ-GPR-pNA (Stefansson, Helgadottir et al. 2010).

### Deactivation of CoV-229E-Luc by Cod Trypsin and Renilla Luciferase Assay

Cod trypsin diluted in Dulbecco's Modified Eagle's medium (DMEM) without Fetal Bovine Serum (FBS), was incubated with CoV-229E-luc (ORF 4 is replaced by Renilla luciferase) per mL at 33° C. for 3 hours. Previously, about 15.000 Huh7 cells were seeded per well into a 96-well plate and incubated at 37° C. and 5% CO₂ in DMEM with 5% FBS. Twenty-four hours later, the cells were washed with PBS, infected with a multiplicity of infection (MOI) of 0.1 with cod trypsin treated CoV-229E-luc. The virus was allowed to adsorb to cells for 2 hours, cells were washed with PBS and cell media. The cells were incubated in DMEM with 5% FBS. Cell viability was assessed by using 10% (v/v) AlamarBlue assay (Fisher Scientific, Inc.) 22 hours post infection. AlamarBlue was incubated at 33° C. and 5% CO₂ for further 24 hours before evaluation in a luminometer at 595 nm. The level of virus replication was determined using the Renilla luciferase Assay kit (Promega, USA) according to the manufactures instructions, using half the recommended substrate concentration, and a luminometer. Logarithmic scale was constructed by calculating log of the luminescence measured.

### Deactivation of CoV-229E by Cod Trypsin

Cod trypsin was evaluated against a challenge virus (human coronavirus, strain 229E, ATCC VR-740) in suspension at two timepoints, 10 and 60 min in a virucidal efficacy suspension test. The test followed the ASTM International test method designated E1052-11 “Standard Test Method to Assess the Activity of Microbicides against Viruses in Suspension”. For each run, two separate dilutions were made. One dilution with 1 part trypsin and 1 part diluent (1× Phosphate buffered saline (PBS)) and the other dilution 1 part trypsin and 3 parts diluent. Each dilution (2.7 ml) was mixed with 0.3 mL of the challenge virus suspension (contained 0% serum) and mixed by vortexing. The reaction mixtures were incubated at 35-37° C. for 10 and 60 min (contact time). After incubation, an aliquot of the reaction mixture was immediately mixed with an equal volume of neutralizer. The neutralizer was minimum essential medium (MEM)+10% FBS+1% polysorbate 80. Sephacryl columns were used to separate the virus from cod trypsin for the test substance and the virus recovery control (see below) after the neutralization by the neutralizer. The quenched sample from the column was serially diluted with medium in tenfold increments and inoculated onto host cells (MRC-5 cells, ATCC CCL-171) to assay for infectious virus. Inoculated plates were incubated at 33±2° C. in 5±1% CO₂ for 5-7 days. After incubation, the cultures were scored for viral infection by determining viral-induced cytopathic effect (CPE). The titer of the virus (log₁₀ TCID₅₀/mL) was calculated using the Spearman-Karber formula (Kärber 1931). The viral load (log 10 TCID50) was calculated by adding the viral titer (log 10 TCID50/mL) to the log 10 (the volume of reaction mixture in mL times the volume correction). The volume correction accounted for the neutralization of the sample post contact time. The log 10 reduction factor was calculated by subtracting the output viral load (log 10) from the input viral load (log 10). The input load was 5.92 but it represents the virus units (log 10 TCID50) recovered after incubating the virus in medium before inoculation (virus recovery control, see below). The output load represents the virus unit (log 10 TCID50) recovered after mixing and incubating the virus with cod trypsin.

Controls included a virus recovery control, neutralizer effectiveness/viral interference control, a cytotoxicity control, cell viability control, a virus stock titer control, and a reference product control. The neutralizer effectiveness/viral interference control was performed to determine if residual active ingredients were present after neutralization and if the neutralized test substance interfered with virus infectivity. A mixture of 1.35 mL of cod trypsin and 1.35 mL of 1×PBS was mixed thoroughly with 0.3 mL of medium (in lieu of the challenge virus), held for 60 min, neutralized and run through a sephacryl column. The quenched sample was divided into 2 portions, one for neutralizer effectiveness/viral interference control, and the other for cytotoxicity control, and each portion serially tenfold diluted. For the neutralizer effectiveness/viral interference control, 0.1 mL of a low titered virus was added to 4.5 mL of each dilution and held for a period equivalent or greater than 60 min. After incubation, the virally spiked dilutions were inoculated onto host cells. For the cytotoxicity control, the sample obtained from the neutralizer effectiveness/viral interference control run was serially diluted and inoculated onto host cells. The condition of the host cells was recorded at the end of the incubation period. For the virus recovery control, 2.7 mL of dilution medium (MEM+5% FBS) was mixed with 0.3 mL of the challenge virus suspension. The mix was held for 60 min, neutralized and run through a sephacryl column as for the test product runs. The quenched sample was serially diluted with dilution medium in tenfold increments and selected dilutions were inoculated onto host cells to assay for infectious virus. For the cell viability control, at least 4 wells were inoculated with media in each assay to demonstrate that cells remained viable and media was sterile throughout the assay. For the virus stock titer control, an aliquot of the virus was serially diluted and inoculated directly onto host cells. This was to demonstrate that the titer of the stock virus was appropriate for use and that the viral infectivity assay was performed appropriately. For the reference product control, a 2.7 mL aliquot of an approximately 1000 ppm NaOCl containing bleach solution was mixed with 0.3 mL of the challenge virus suspension. The mix was held for the two contact times and then neutralized. The quenched sample was serially diluted with dilution medium in tenfold increments and selected dilutions were inoculated onto host cells to assay for infectious virus. The viral stock titer control for each assay confirmed that the appropriate titer was used in the experiment and sufficient amount of virus was recovered for the virus recovery control. No virus was detected in the cell viability control wells, the cells remained viable and the media was sterile. Virus was detected in all the neutralizer effectiveness/viral interference control wells. Cytotoxicity was not detected at any dilution or cell line tested. Viral-induced CPE was distinguishable from uninfected cells. Thus, all the controls met the criteria for a valid test. The reference test substance, 1000 ppm NaOCl, had a log reduction of a 4.31 for all viruses tested.

The virucidal efficacy suspension test was carried out by an independent testing laboratory; Microbac Laboratories, Inc., 105 Carpenter Drive, Sterling, Va. 20164, USA.

### Viral Protein Analysis of Cod Trypsin Treated CoV-229E-Luc

Cod trypsin diluted in DMEM (without FBS) was mixed with CoV-229E-luc virus. The mixture was incubated at 33° C. for 2 hours. After incubation, Laemmli buffer (4×) was added and the mixture incubated for 5 min at 95° C. The sample was loaded on a 10% sodium dodecyl sulfate polyacrylamide gel electrophoresis (SDS-PAGE) gel (Bachofen, Bollinger et al. 2013) and Western blot analysis was conducted, see below.

Treatment of Recombinant CoV-229E S1 Spike Protein with Cod Trypsin

Cod trypsin diluted in serum free DMEM, was used to treat recombinant CoV-229E S1 spike protein at a final concentration of 86 μg/mL. The mixture was incubated at 33° C. for 2 hours. After incubation, Laemmi buffer (4×) was added and incubated for 5 min at 95° C. to inactivate the enzyme. The sample was loaded on a 10% SDS-PAGE gel (Bachofen, Bollinger et al. 2013) and Western blot analysis was conducted, see below. Recombinant CoV-229E S1 spike protein was obtained from Prof. Dr. Volker Thiel at the Institute of Virology and Immunology, University of Bern, Switzerland.

### Western Blot Analysis

For Western blot analysis, SDS-PAGE (10%) was performed and the proteins were transferred onto a nitrocellulose membrane. After transfer, the membrane was blocked with PBS containing 5% milk and 0.5% Tween-20 for 60 min at room temperature (RT). Primary antibody (anti-coronavirus polyclonal goat antibodies produced from NP-40-dispersed CoV-229E (Wentworth and Holmes 2001)) was diluted 1:1000 in PBS-Tween (PBS-T) with 0.5% milk and incubated overnight with gentle agitation at 4° C. The membrane was washed with PBS followed by incubation with secondary antibody (anti-goat IgG conjugated to horse radish peroxidase, 1:1000 in 0.5% milk) for 60 min at RT. The immunoblot was developed using a charge-coupled device (CCD) camera.

Treatment of Recombinant SARS-CoV Spike Protein with Trypsin ZT or Trypsin I

A recombinant Spike Protein (Beijing02) (SARS-CoV) (eEnzyme, catalog number: SS-001-005P) (0.1 mg/mL) was incubated with trypsin ZT or trypsin I at 33° C. for 10 min in 17 mM Tris, 12.5 mM CaCl₂, 2.5 mM ethanolamine at pH 8.5. The samples were incubated for 10 min at 95° C. in 4×LDS sample buffer. As a positive control, spike protein was incubated at 33° C. for 10 min without trypsin isoenzymes. The mixture was run on a 12% SDS-PAGE gel (NuPAGE Bis-Tris, Novex by life technologies) using MOPS-SDS running buffer. The gel was stained with Coomassie blue (PageBlue Protein Staining Solution, Thermo Fisher Scientific) and imaged on an Odyssey infrared imaging system.

### Results

### Deactivation of Coronavirus by Cod Trypsin

To study the ability of cod trypsin in reducing the infectivity of coronavirus, an assay using CoV-229E-luc utilizing Renilla luciferase was used (van den Worm, Eriksson et al. 2012). Coronavirus was incubated (180 min) with or without cod trypsin and placed on human liver cells (Huh7). After the viral adsorption period, cells were incubated for two days and the level of coronavirus infection measured (see Methods). The level of infection was measured using the Renilla luciferase assay (FIG. 1).

Treatment with 1.4 and 2.8 U/mL cod trypsin lowered the level of coronaviral infection by about 3 log units compared to control (untreated coronavirus) (FIG. 1).

To further test the ability of cod trypsin to deactivate coronavirus, an experiment was performed using a standardized virucidal efficacy suspension test with coronavirus 229E. The coronavirus was incubated for 10 or 60 min with cod trypsin at a concentration of 1.22 U/mL or 2.44 U/mL, in duplicate. Samples from each incubation were titrated with the 50% Tissue Culture Infectious Dose (TCID₅₀) endpoint assay (see Methods). The log₁₀ reduction factor was calculated and the mean reported (FIG. 2).

Cod trypsin treatment of coronavirus resulted in greater than 4 log reduction in virus infectivity after incubation for 60 min at both concentrations. With incubation for 10 min, 2.5 and 3 log reduction was observed at 1.22 U/mL and 2.44 U/mL, respectively.

### Degradation of Viral Proteins in Cod Trypsin Treated Coronavirus Samples

The ability of cod trypsin to degrade coronavirus proteins in samples containing infectious coronavirus was tested. A stock solution of coronavirus was treated with cod trypsin at a concentration of 1.4-5.6 U/mL for 3 hours at 33° C. and the samples subjected to Western blot analysis (FIG. 3). As can be seen in FIG. 3, cod trypsin is able to degrade coronavirus proteins at a concentration of 1.4-5.6 U/mL in 3 hours. The size of the protein recognized by the antibody (50 kDa) matches the size of CoV-229E nucleocapsid protein (Lo, Lin et al. 2013).

To further study the ability of cod trypsin to degrade proteins from coronavirus, recombinant CoV-229E spike protein was incubated with different concentrations of cod trypsin at 33° C. for 2 hours and subjected to Western blot analysis (FIG. 4).

As can be seen in FIG. 4, cod trypsin degrades the recombinant coronavirus spike protein at all concentrations tested.

It was of interest to test the ability of the cod trypsin isoenzymes trypsin ZT and trypsin I to degrade recombinant SARS-CoV spike protein. Both isoenzymes are found in the cod trypsin isolate used in the study. In FIG. 5 the SARS-CoV spike protein is seen around 180 kDa. The spike protein is degraded at both concentrations tested (0.35 and 0.7 U/mL) by trypsin ZT and trypsin I in 10 min at 33° C.

### Discussion

In this study, it was demonstrated that cod trypsin reduced the level of infection of coronavirus by more than 4 log units in 60 min and up to 3 log units in only 10 min (FIG. 2). The ability of cod trypsin to deactivate coronavirus was also established using a coronavirus (CoV-229E-luc) where the viral titer was monitored with a Renilla luciferase assay (FIG. 1). The deactivating efficacy of cod trypsin against coronavirus is thought to be based on its capacity to degrade coronavirus spike proteins. This would diminish the ability of the coronavirus to bind to human cell receptors important for infection (Lim, Ng et al. 2016, Park, Li et al. 2016). In support of this mode of action, degradation of coronavirus proteins by cod trypsin was analyzed. CoV-229E-luc virus treated with cod trypsin was subjected to Western blot analysis using a polyclonal antibody raised against CoV-229E (FIG. 3). The results clearly demonstrate degradation of a protein in the size range of 50 kDa that correlates well with that of nucleocapsid protein (Lo, Lin et al. 2013). Furthermore, recombinant CoV-229E spike protein in a purified form was degraded by cod trypsin as seen in FIG. 4. The data obtained on coronavirus protein degradation are based on incubation of the virus with cod trypsin for 2 hours (FIG. 3). It is suspected that the spike proteins are most susceptible to cod trypsin degradation as they are located on the surface of the coronavirus. Based on our findings, incubation of coronavirus with cod trypsin starts with degradation of spike proteins (FIG. 4 and FIG. 5). However, after an extensive incubation period the nucleocapsid proteins are degraded (FIG. 3).

The benzamidine purified cod trypsin fraction used in the study contains different cod trypsin isoenzymes such as trypsin I, trypsin ZT and trypsin X (see above). All these trypsin isoenzymes cleave at arginine and lysine but trypsin I and trypsin ZT have been shown to have different subsite specificity (unpublished data; Sandholt G B, Stefansson B, Gudmundsdottir Á). Therefore, it was of interest to test if these isoenzymes can cleave spike proteins from coronaviruses. For this purpose, recombinant spike protein from SARS-CoV was selected. This is of high importance as MERS and SARS have high mortality rates and are on the top of WHO's list of disease priorities needing urgent research due to the high likelihood of causing a major epidemic (Gralinski and Baric 2015, Mackay and Arden 2015).

Based on SDS-PAGE analysis, recombinant SARS-CoV spike protein was effectively degraded by both cod trypsin I and trypsin ZT at low concentrations in only 10 min (FIG. 5). The findings indicate that SARS coronavirus is likely to be susceptible to deactivation by cod trypsin at low concentrations. This suggests that the different coronaviruses that cause infection in humans could be deactivated by cod trypsin. The fact that the trypsin isoenzymes were effective in cleaving SARS-CoV spike protein in 10 min (FIG. 5) aligns well with the results in FIG. 2 where cod trypsin lowered the level of infection of coronavirus in 10 min.

The results presented in this study on the ability of cod trypsin to deactivate coronaviruses extend the findings of other studies that show efficacy of cod trypsin against different respiratory viruses (Gudmundsdottir, Hilmarsson et al. 2013). Interestingly, lysine and arginine rich amino acid sequences are frequently found in viral proteins (Suzuki, Orba et al. 2010, Jiang, Cun et al. 2012, Gallaher and Garry 2015). These positively charged basic amino acid residues are mainly exposed to the protein surface and are important for protein stability by forming electrostatic interactions (Sokalingam, Raghunathan et al. 2012). The fact that arginine and lysine residues are common in viral proteins can explain the ability of cod trypsin to efficiently cleave the coronavirus spike proteins tested in this study (FIG. 4 and FIG. 5).

In conclusion, the results identify that the ability of cod trypsin to deactivate coronaviruses via degradation of surface spike proteins that prevent adhesion of coronaviruses to cells.

### REFERENCES

Asgeirsson, B. and P. Cekan (2006). “Microscopic rate-constants for substrate binding and acylation in cold-adaptation of trypsin I from Atlantic cod.” FEBS Lett 580(19): 4639-4644.
Asgeirsson, B., J. W. Fox and J. B. Bjarnason (1989). “Purification and characterization of trypsin from the poikilotherm Gadus morhua.” Eur J Biochem 180(1): 85-94.
Ásgeirsson & Bjarnason (1991) Structural and kinetic properties of chymotrypsin from Atlantic cod (Gadus morhua). Comparison with bovine chymotrypsin. Comp. Biochem. Physiol. B 998:327-335
Bachofen, C., B. Bollinger, E. Peterhans, H. Stalder and M. Schweizer (2013). “Diagnostic gap in Bovine viral diarrhea virus serology during the periparturient period in cattle.” J Vet Diaqn Invest 25(5): 655-661.
Belouzard, S., J. K. Millet, B. N. Licitra and G. R. Whittaker (2012). “Mechanisms of coronavirus cell entry mediated by the viral spike protein.” Viruses 4(6): 1011-1033.
Bradburne, A. F., M. L. Bynoe and D. A. Tyrrell (1967). “Effects of a “new” human respiratory virus in volunteers.” Br Med J 3(5568): 767-769.
Fehr, A. R. and S. Perlman (2015). “Coronaviruses: an overview of their replication and pathogenesis.” Methods Mol Biol 1282:1-23.
Fornbacke, M. and M. Clarsund (2013). “Cold-adapted proteases as an emerging class of therapeutics.” Infect Dis Ther 2(1): 15-26.
Gallaher, W. R. and R. F. Garry (2015). “Modeling of the Ebola virus delta peptide reveals a potential lytic sequence motif.” Viruses 7(1): 285-305.
Graham, R. L. and R. S. Baric (2010). “Recombination, reservoirs, and the modular spike: mechanisms of coronavirus cross-species transmission.” J Virol 84(7): 3134-3146.
Gralinski, L. E. and R. S. Baric (2015). “Molecular pathology of emerging coronavirus infections.” J Pathol 235(2): 185-195.
Green & Sambrook (2012) Molecular Cloning: a Laboratory Manual, 4th edition, Cold Spring Harbor Laboratory Press
Gudmundsdottir, A., E. Gudmundsdottir, S. Oskarsson, J. B. Bjarnason, A. K. Eakin and C. S. Craik (1993). “Isolation and characterization of cDNAs from Atlantic cod encoding two different forms of trypsinogen.” Eur J Biochem 217(3): 1091-1097.
Gudmundsdottir, A., H. Hilmarsson and B. Stefansson (2013). “Potential use of Atlantic cod trypsin in biomedicine.” Biomed Res Int 2013: 749078.
Heikkinen, T. and A. Jarvinen (2003). “The common cold.” Lancet 361(9351): 51-59.
Hull, D., P. Rennie, A. Noronha, C. Poore, N. Harrington, V. Fearnley and D. Passali (2007). “Effects of creating a non-specific, virus-hostile environment in the nasopharynx on symptoms and duration of common cold.” Acta Otorhinolaryngol Ital 27(2): 73-77.
Jiang, J., W. Cun, X. Wu, Q. Shi, H. Tang and G. Luo (2012). “Hepatitis C virus attachment mediated by apolipoprotein E binding to cell surface heparan sulfate.” J Virol 86(13): 7256-7267.
Jonsdottir, H. R. and R. Dijkman (2016). “Coronaviruses and the human airway: a universal system for virus-host interaction studies.” Virology journal 13(1): 24.
Kärber, G. (1931). “Beitrag zur kollektiven Behandlung pharmakologischer Reihenversuche.” Naunyn-Schmiedeberg's Archives of Pharmacology 162(4): 480-483.
Kraaijeveld, C. A, S. E. Reed and M. R. Macnaughton (1980). “Enzyme-linked immunosorbent assay for detection of antibody in volunteers experimentally infected with human coronavirus strain 229 E.” J Clin Microbiol 12(4): 493-497.
Lim, X. Y., L. Y. Ng, P. J. Tam and X. D. Liu (2016). “Human Coronaviruses: A Review of Virus-Host Interactions.” Diseases 4(3).
Lo, Y. S., S. Y. Lin, S. M. Wang, C. T. Wang, Y. L. Chiu, T. H. Huang and M. H. Hou (2013). “Oligomerization of the carboxyl terminal domain of the human coronavirus 229E nucleocapsid protein.” FEBS Lett 587(2): 120-127.
Mackay, L. M. and K. E. Arden (2015). “MERS coronavirus: diagnostics, epidemiology and transmission.” Virol J 12: 222.
Meziere et al. (1997) In vivo T helper cell response to retro-inverso peptidomimetics. J. Immunol. 159, 3230-3237
Park, J. E., K. Li, A. Barlan, A. R. Fehr, S. Perlman, P. B. McCray, Jr. and T. Gallagher (2016). “Proteolytic processing of Middle East respiratory syndrome coronavirus spikes expands virus tropism.” Proc Natl Acad Sci USA 113(43): 12262-12267.
Sokalingam, S., G. Raghunathan, N. Soundrarajan and S. G. Lee (2012). “A study on the effect of surface lysine to arginine mutagenesis on protein stability and structure using green fluorescent protein.” PLoS One 7(7): e40410.
Spilliaert, R. and A. Gudmundsdottir (1999). “Atlantic Cod Trypsin Y-Member of a Novel Trypsin Group.” Mar Biotechnol (NY) 1(6): 598-607.
Stefansson, B., L. Helgadottir, S. Olafsdottir, A. Gudmundsdottir and J. B. Bjarnason (2010). “Characterization of cold-adapted Atlantic cod (Gadus morhua) trypsin 1—kinetic parameters, autolysis and thermal stability.” Comp Biochem Physiol B Biochem Mol Biol 155(2):186-194.
Stefansson, B., G. B. Sandholt and A. Gudmundsdottir (2017). “Elucidation of different cold-adapted Atlantic cod (Gadus morhua) trypsin X isoenzymes.” Biochim Biophys Acta 1865(1):11-19.
Suzuki, T., Y. Orba, Y. Okada, Y. Sunden, T. Kimura, S. Tanaka, K. Nagashima, W. W. Hall and H. Sawa (2010). “The human polyoma JC virus agnoprotein acts as a viroporin.” PLoS Pathog 6(3): e1000801.
Thompson et al. (1994) CLUSTAL W: improving the sensitivity of progressive multiple sequence alignment through sequence weighting, position-specific gap penalties and weight matrix choice. Nuc. Acid Res. 22:4673-4680
Thorsett et al. (1983) Dipeptide mimics. Conformationally restricted inhibitors of angiotensin-converting enzyme Biochem. Biophys. Res. Comm. 111:166
van den Worm, S. H., K. K. Eriksson, J. C. Zevenhoven, F. Weber, R. Zust, T. Kuri, R. Dijkman, G. Chang, S. G. Siddell, E. J. Snijder, V. Thiel and A. D. Davidson (2012). “Reverse genetics of SARS-related coronavirus using vaccinia virus-based recombination.” PLoS One 7(3): e32857.
Veber et a. (1978) Conformationally restricted bicyclic analogs of somatostatin Proc. Natl. Acad. Sci. USA 75:2636
Wentworth, D. E. and K. V. Holmes (2001). “Molecular Determinants of Species Specificity in the Coronavirus Receptor Aminopeptidase N (CD13): Influence of N-Linked Glycosylation.” J Virol 75(20): 9741-9752.
WHO. “Emergencies preparedness, response.” Retrieved 2 Feb. 2017, from http://www.who.int/csr/don/archive/disease/coronavirus_infections/en.
Remington's Pharmaceutical Sciences, 18th edition, A. R Gennaro, Ed., Mack Publishing Company (1990)
Handbook of Pharmaceutical Excipients, 3rd edition, A. Kibbe, Ed., Pharmaceutical Press (2000)

1. A method of treating or preventing coronavirus infection in a subject comprising administering to said subject a polypeptide having protease activity.

2. The method according to claim 1 wherein the coronavirus infection is an infection of the respiratory tract and/or of the gastrointestinal tract.

3. The method according to claim 1 wherein the coronavirus infection is selected from the group consisting of common cold, pneumonia, pneumonitis, bronchitis, severe acute respiratory syndrome (SARS), Middle East respiratory syndrome (MERS), sinusitis, otitis or pharyngitis.

4. The method according to claim 3 wherein the coronavirus infection is the common cold.

5. The method according to claim 1 wherein the coronavirus is selected from the group consisting of:
(a) alphacoronavirus;
(b) betacoronavirus;
(c) gammacoronavirus; and
(d) deltacoronavirus.

6. The method according to claim 1 wherein the coronavirus is a human coronavirus.

7. The method according to claim 6 wherein the human coronavirus is selected from the group consisting of:
(a) human coronavirus 229E;
(b) human coronavirus OC43;
(c) Severe Acute Respiratory Syndrome coronavirus (SARS-CoV)
(d) human Coronavirus NL63 (HCoV-NL63, New Haven coronavirus);
(e) human coronavirus HKU1; and
(f) Middle East respiratory syndrome coronavirus (MERS-CoV).

8. The method according to claim 1 wherein the subject is a mammal, for example a human.

9. The method according to claim 1 wherein the polypeptide having protease activity is selected from the group consisting of serine proteases, threonine proteases, cysteine proteases, aspartate proteases, glutamic acid proteases and metalloproteases.

10. The method according to claim 9 wherein the protease is a serine protease.

11. The method according to claim 10 wherein the protease is a trypsin or chymotrypsin, or a component of a mixture thereof.

12. A polypeptide for use according to any one of the preceding claims wherein the polypeptide having protease activity is cold-adapted.

13. The method according to claim 1 wherein the polypeptide is naturally occurring.

14. The method according to claim 13 wherein the polypeptide is a marine serine protease.

15. The method according to claim 14 wherein the marine serine protease is obtained or obtainable from cod, pollock, salmon or krill.

16. The method according to claim 15 wherein the marine serine protease is obtained or obtainable from Atlantic cod.

17. The method according to claim 14 wherein the marine serine protease is a trypsin, for example trypsin I.

18. The method according to claim 1 wherein the polypeptide is trypsin I, trypsin X or trypsin ZT from Atlantic cod.

19. The method according to claim 1 wherein the activity of the trypsin ranges from 1 U/mg to 1 U/g of the polypeptide, for example between 50 U/mg and 500 U/mg of the polypeptide.

20. The method according to claim 1 wherein the polypeptide is non-naturally occurring.

21. The method according to claim 1 wherein the polypeptide comprises or consists of an amino acid sequence of any one of SEQ ID NOs: 1 to 12, or a fragment, variant, derivative or fusion thereof (or a fusion of said fragment, variant or derivative) which retains the protease activity of said amino acid sequence.

22. The method according to claim 21 wherein the polypeptide comprises or consists of an amino acid sequence selected from any one of SEQ ID NOs: 1 to 12.

23. The method according to claim 1 wherein the polypeptide comprises or consists of a fragment of the amino acid sequence according to SEQ ID NOs: 1 to 12.

24. A polypeptide for use according to claim 23 wherein the fragment comprises or consists of at least 15 contiguous amino acid of any one of SEQ ID NOs: 1 to 12, for example at least 16, 17, 18, 19, 20, 30, 40, 50, 60, 70, 80, 90, 100, 110, 120, 130, 140, 150, 160, 170, 180, 190, 200, 210, 220, 230 or 240 contiguous amino acids of any one of SEQ ID NOs: 1 to 12.

25. The method according to any one of claims 1 to 21 wherein the polypeptide comprises or consists of a variant of the amino acid sequence according to any one of SEQ ID NOs: 1 to 12.

26. The method according to claim 25 wherein the variant is a non-naturally occurring variant.

27. The method according to claim 25 wherein the variant has an amino acid sequence which has at least 50% identity with the amino acid sequence according to any one of SEQ ID NOs: 1 to 12, or a fragment thereof, for example at least 55%, 60%, 65%, 70%, 75%, 80%, 90%, 95%, 96%, 97%, 98% or at least 99% identity.

28. The method according to claim 1 wherein the polypeptide is between 150 and 250 amino acids in length, for example between 200 and 250, between 210 and 240, between 220 and 230, or between 220 and 225 amino acids in length.

29. The method according to claim 1 wherein the polypeptide is a recombinant polypeptide.

30. The method according to claim 1 wherein the polypeptide is provided in an osmotically active solution.

31. The method according to claim 1 wherein the polypeptide is administered in combination with glycerol and a buffer.

32. The method according to claim 1 wherein the polypeptide is provided in a form suitable for delivery to the oropharynx.

33. The method according to claim 1 wherein the polypeptide is provided in a mouth spray, lozenge, pastille, tablet, syrup or chewing gum.

34. The method according to claim 1 wherein the polypeptide is for use in combination with one or more additional active agents.

35. The method according to claim 34 wherein the additional active agents are selected from the group consisting of antimicrobial agents (including antibiotics, antiviral agents and anti-fungal agents), anti-inflammatory agents (including steroids and non-steroidal anti-inflammatory agents) and antiseptic agents.

36. The method according claim 35 wherein the one or more antimicrobial agents are antibiotics selected from the group consisting of penicillins, cephalosporins, fluoroquinolones, aminoglycosides, monobactams, carbapenems and macrolides.

37. A method the preparation of a medicament comprising formulating a polypeptide according to claim 1 in a pharmaceutical composition.

38. The method according to claim 37 wherein the polypeptide is a trypsin or chymotrypsin, or a component of a mixture thereof.

39. The method according to claim 37 wherein the polypeptide comprises or consists of an amino acid sequence of any one of SEQ ID NOS: 1 to 12, or a fragment, variant, derivative or fusion thereof (or a fusion of said fragment, variant or derivative) which retains the trypsin activity of said amino acid sequence.

40. The method according to claim 39 wherein the polypeptide comprises or consists of an amino acid sequence of any one of SEQ ID NOS: 1 to 121.

41. The method according to claim 37 wherein the coronavirus infection is selected from the group consisting of common cold, pneumonia, pneumonitis, bronchitis, severe acute respiratory syndrome (SARS), Middle East respiratory syndrome (MERS), sinusitis, otitis or pharyngitis.

42-49. (canceled)
